(19) United States
(12) Patent Application Publication
(10) Pub. No.: US 2026/0098244 A1
(43) Pub. Date: Apr. 9, 2026

(54) DEVICES, CHROMATOGRAPHY COLUMNS, METHODS FOR AUTOMATED CHROMATOGRAPHY IN PARALLEL AND METHODS FOR PURIFICATION OF EXTRACELLULAR VESICLES

(71) Applicants: President and Fellows of Harvard College, Cambridge, MA (US); The Brigham and Women's Hospital, Inc., Boston, MA (US)

(72) Inventors: Dmitry Ter-Ovanesyan, Cambridge, MA (US); Tal Gilboa, Cambridge, MA (US); Bogdan Budnik, Boston, MA (US); David Kalish, Cambridge, MA (US); George M. Church, Cambridge, MA (US); David R. Walt, Boston, MA (US)

(73) Assignee: President and Fellows of Harvard College, Cambridge, MA (US)

(21) Appl. No.: 19/265,231

(22) Filed: Jul. 10, 2025

Related U.S. Application Data
(63) Continuation of application No. PCT/US2024/011933, filed on Jan. 18, 2024.
(60) Provisional application No. 63/439,670, filed on Jan. 18, 2023.
(60) Provisional application No. 63/543,146, filed on Oct. 9, 2023.

(51) Int. Cl. C12N 5/078; B01D 15/18; B01D 15/34; B01D 15/36

(52) U.S. Cl. CPC C12N 5/0634; B01D 15/1885; B01D 15/34; B01D 15/362

(57) ABSTRACT

The technology described herein is directed to devices and methods for automated chromatography analysis of multiple samples in parallel. The devices include a pump, a computing device, and a stand having an upper portion including columns containing a resin and a bottom portion including wells or tubes. The invention also provides disclosures for chromatography columns and methods for purification of extracellular vesicles from a biological sample.

### RELATED APPLICATIONS

This application is a continuation of International Application No. PCT/US/2024/011933, filed on Jan. 18, 2024, which claims the benefit of priority to U.S. Provisional Application No. 63/439,670, filed on Jan. 18, 2023, and U.S. Provisional Application No. 63/543,146, filed on Oct. 9, 2023. The entire contents of each of the foregoing applications are incorporated herein by reference.

### TECHNICAL FIELD

Described herein are devices and methods related to chromatography, such as devcies and methods for automated analysis of multiple samples in parallel. The invention also provides disclosures related to novel chromatography columns and methods for purification of extracellular vesicles from a biological sample.

### BACKGROUND OF THE INVENTION

Extracellular vesicles (EVs) are membrane vesicles released by all cells that contain cargo from their cell of origin. EVs represent a novel class of biomarkers in biofluids such as plasma. As EVs are much less abundant than free proteins and lipoproteins, isolating them without the co-isolation of these contaminants remains highly challenging. This is particularly the case for separating EVs and lipoproteins, as these two classes of particles have overlapping size ranges. Developing EV isolation methods is also challenging due to the inability of most methods, such as the commonly used Nanoparticle Tracking Analysis (NTA), to differentiate between EVs and similarly-sized (but considerably more abundant) lipoproteins such as low-density lipoprotein (LDL) and very low-density lipoprotein (VLDL). Thus, it is difficult to compare EV isolation methods without suitable techniques to quantify EV yield and lipoprotein quantification.

Accordingly, there remains a need in the art for a highly sensitive and efficient method for purifying EVs from biological fluids.

### SUMMARY OF THE INVENTION

Accordingly, in one aspect, the present invention provides a device for automated chromatography of multiple samples in parallel.

In some embodiments, the device includes a stand including a frame, an upper portion attached to the frame, and a bottom portion connected to the frame. In some embodiments, the upper portion includes a column containing a resin and configured to receive a biological sample. In some embodiments, the bottom portion includes a row of openings, each opening configured to house a tube. In some embodiments, the bottom portion may be positioned such that the row of openings is positioned to receive the biological sample from the upper portion.

In some embodiments, the device includes a pump configured to deliver the biological sample. In some embodiments, the device includes a computer device configured to control the flow of the biological sample from the pump. In some embodiments, the pump is a syringe pump.

In some embodiments, the bottom portion is configured to house at least eight tubes. In some embodiments, the bottom portion is configured to house at least twenty-four tubes. In some embodiments, the bottom portion is configured to house at least ninety-six tubes. In some embodiments, the bottom portion is slideably connected to the frame.

In some embodiments, each tube is sized to receive a sample ranging from half a milliliter to 50 milliliters.

In some embodiments, the column comprises a size exclusion chromatography resin, a cation-exchange resin, and/or size exclusion beads capable of capturing molecules smaller than about 700 kDa.

In some embodiments, the size exclusion chromatography resin comprises a Sepharose™ CL-6B resin.

In some embodiments, the cation exchange chromatography resin comprises a Fractogel® EMD-SO₃⁻ resin.

In some embodiments, the size-exclusion beads comprise Capto™ Core 700 beads.

In some embodiments, the tube is an Eppendorf tube.

In some embodiments, the biological sample is a plasma sample.

In some embodiments, the bottom portion is slideably connected to the frame.

In another aspect, the present invention provides a method of automating chromatography of multiple samples in parallel.

In some embodiments, the method includes providing a device including a stand including a frame, an upper portion attached to the frame, and a bottom portion connected to the frame. In some embodiments, the upper portion includes a column containing a resin and configured to receive a biological sample. In some embodiments, the bottom portion comprising a row of openings, each opening configured to house a tube. In some embodiments, the bottom portion may be positioned such that the row of openings is positioned to receive the biological sample from the upper portion. The method further inclues initiating a flow of the biological sample into the device. The sample includes extracellular vesicles.

In some embodiments, the method further includes collecting fractions containing extracellular vesicles from the column, thereby purifying the extracellular vesicles.

In some embodiments, the flow is provided by a pump. In some embodiments, operation of the pump is controlled by a computer device. In some embodiments, the pump is a syringe pump.

In some embodiments, the bottom portion is configured to house at least eight tubes. In some embodiments, the bottom portion is configured to house at least twenty-four tubes. In some embodiments, the bottom portion is configured to house at least ninety-six tubes.

In some embodiments, each tube is sized to receive a sample ranging from half a milliliter to 50 milliliters.

In some embodiments, the column comprises a size exclusion chromatography resin, a cation-exchange resin, and/or size exclusion beads capable of capturing molecules smaller than about 700 kDa.

In some embodiments, the size exclusion chromatography resin comprises a Sepharose™ CL-6B resin.

In some embodiments, the cation exchange chromatography resin comprises a Fractogel® EMD-SO₃⁻ resin.

In some embodiments, the size-exclusion beads comprise Capto™ Core 700 beads.

In some embodiments, the tube is an Eppendorf tube.

In some embodiments, the biological sample is a plasma sample.

In some embodiments, the bottom portion is slideably connected to the frame.

In some embodiments, the present invention provides a chromatography column for purifying extracellular vesicles from a biological sample comprising: (a) a size exclusion chromatography resin, (b) a cation exchange chromatography resin; and (c) size-exclusion beads capable of capturing molecules smaller than about 700 kDa; wherein the size exclusion chromatography resin is placed at a top layer of the column; and wherein the cation exchange chromatography resin and the size-exclusion beads are mixed and the mixture is placed at a bottom layer of the column.

In some embodiments, the size exclusion chromatography resin comprises a Sepharose™ CL-6B resin.

In some embodiments, the cation exchange chromatography resin comprises a Fractogel® EMD-SO₃⁻ resin.

In some embodiments, the size-exclusion beads comprise Capto™ Core 700 beads.

In some embodiments, the top layer of the column comprises about 5 mL, about 6 mL, about 7 mL, about 8 mL, about 9 mL, or about 10 mL of the size exclusion chromatography resin.

In some embodiments, the top layer of the column comprises about 10 mL of the size exclusion chromatography resin.

In some embodiments, the bottom layer of the column comprises about 1 mL, about 2 mL, or about 4 mL of the mixture of the cation exchange chromatography resin and the size-exclusion beads.

In some embodiments, the bottom layer of the column comprises about 2 mL of the mixture of the cation exchange chromatography resin and the size-exclusion beads.

In some embodiments, the cation exchange chromatography resin and the size-exclusion beads are mixed at a ratio of about 1:4, about 1:2, about 2:1, or about 4:1 by volume.

In some embodiments, the cation exchange chromatography resin and the size-exclusion beads are mixed at a ratio of about 2:1 by volume.

In some embodiments, the biological sample is obtained from a subject. In some embodiments, the biological sample is a plasma sample. In some embodiments, the biological sample is a cerebrospinal fluid (CSF) sample.

In another aspect, the present invention provides a method of purifying extracellular vesicles from a biological sample, the method comprising: (a) providing a chromatography column of the invention as described herein, (b) introducing a biological sample comprising extracellular vesicles into the column, and (c) collecting fractions containing extracellular vesicles from the column, thereby purifying the extracellular vesicles.

### BRIEF DESCRIPTION OF THE DRAWINGS

FIG. 1 schematically depicts a perspective view of a chromatography stand.

FIG. 2 depicts a photograph of a chromatography stand connected to a computing device and a pump.

FIG. 3 depicts Simoa comparisons of various assays when chromatography was performed on samples manually and automatically.

FIGS. 4A-4D are related to an embodiment of a chromatography stand including a slideable bottom portion. FIG. 4A schematically depicts a perspective view of a chromatography stand including a slideable bottom portion positioned under an upper portion. FIG. 4B schematically depicts a side view of a chromatography stand including a slideable bottom portion positioned under an upper portion. FIG. 4C schematically depicts a perspective view of a chromatography stand including a slideable bottom portion slid out from under an upper portion. FIG. 4D schematically depicts a side view of a chromatography stand including a slideable bottom portion slid out from under an upper portion.

FIGS. 5A-5B depict the validation of ApoB100 Simoa assay. FIG. 5A depicts the calibration curve for the Simoa ApoB100 assay. FIG. 5B depicts the dilutions of human plasma (from three different individuals) to confirm dilution linearity. Error bars represent the standard deviation from two technical replicates.

FIGS. 6A-6C depict purification of extracellular vesicles by size exclusion chromatography (SEC) of plasma using different resins. FIG. 6A depicts the relative levels of CD9, CD63, CD81, ApoB100, and Albumin measured by Simoa after SEC in each fraction using either Sepharose CL-2B (blue), Sepharose CL-4B (green), or Sepharose CL-6B (red) resin. Levels of each protein were normalized to the highest measurement. FIG. 6B depicts the recovery of EVs calculated for Sepharose CL-2B (fractions 9-12), Sepharose CL-4B (fractions 7-10), or Sepharose CL-6B (fractions 7-10). Simoa measurements in the designated fractions for CD9, CD63, and CD81 were taken as a ratio relative to measurements of these proteins from diluted plasma and these three ratios were then averaged to calculate recovery. FIG. 6C depicts the purity of EVs with respect to lipoproteins or free proteins calculated by dividing relative EV yield (the average of the ratios of CD9, CD63, and CD81) by levels of ApoB100 (top) or Albumin (bottom). Error bars represent the standard deviation of four replicates from each column.

FIG. 7 depicts the separation of EVs, lipoproteins, and free proteins from plasma using density gradient centrifugation. CD9, CD63, CD81, Albumin and ApoB100 were measured by Simoa (in individual 1 mL fractions collected from the top) after density gradient centrifugation of plasma using an iodixanol gradient. Error bars represent the standard deviation of two replicates of each measurement.

FIGS. 8A-8E depict the comparison of novel columns for EV isolation using electron microscopy and Simoa. FIG. 8A depicts the shematic of the columns being compared: size exclusion chromatography column comprised of 10 mL Sepharose CL-6B, dual mode chromatography (DMC) columns comprised of 10 mL Sepharose CL-6B SEC resin atop 2 mL Fractogel cation exchange resin, tri-mode chromatography (TMC) columns comprised of 10 mL Sepharose CL-6B SEC resin atop 2 mL 2:1 ratio of 2 mL Fractogel cation exchange resin to Capto Core multimodal chromatography resin. FIG. 8B depicts the electron microscopy of EVs isolated from plasma using SEC (left), DMC (middle), or TMC (right) columns. EVs indicated with red arrows. FIG. 8C depicts the EV recovery calculated for EV isolation from plasma for SEC (fractions 7-10), DMC (fractions 9-12), or TMC (fractions 9-12). Simoa measurements in the designated fractions for CD9, CD63, and CD81 were taken as a ratio relative to measurements of these proteins from diluted plasma and these three rations are then averaged to calculate recovery. FIG. 8D depicts the purity of EVs with respect to free proteins determined by dividing relative EV yield (the average of the ratios of CD9, CD63, and CD81) by levels of Albumin in each condition. FIG. 8E depicts the purity of EVs with respect to lipoproteins determined by dividing relative EV yield (the average of the ratios of CD9, CD63, and CD81) by levels of ApoB100 in each condition. Error bars represent the standard deviation of four replicates from each column.

FIGS. 9A-9E depict the comparison of various columns for EV isolation: Sepharose CL-6B, dual mode chromatography (DMC) columns comprised of 10 mL Sepharose CL-6B SEC resin atop 2 mL Fractogel cation exchange resin, tri-mode chromatography (TMC) columns comprised of 10 mL Sepharose CL-6B SEC resin atop Fractogel cation exchange resin to Capto Core multimodal chromatography resin at different ratios (1:4, 1:2, 2:1, 4:1) and different volume (1 mL, 2 mL, and 4 mL). FIG. 9A depicts the relative level of Albumin measured by Simoa. FIG. 9B depicts the relative level of ApoB100 measured by Simoa. FIG. 9C depicts the relative level of CD9 measured by Simoa. FIG. 9D depicts the relative level of CD63 measured by Simoa. FIG. 9E depicts the relative level of Albumin measured by CD81.

FIGS. 10A-10C depict the comparison of various columns for EV isolation: Sepharose CL-6B, dual mode chromatography (DMC) columns comprised of 10 mL Sepharose CL-6B SEC resin atop 2 mL Fractogel cation exchange resin, tri-mode chromatography (TMC) columns comprised of 10 mL Sepharose CL-6B SEC resin atop Fractogel cation exchange resin to Capto Core multimodal chromatography resin at different ratios (1:4, 1:2, 2:1, 4:1) and different volume (1 mL, 2 mL, and 4 mL). FIG. 10A depicts the purity of EVs with respect to lipoproteins determined by dividing relative EV yield (the average of the ratios of CD9, CD63, and CD81) by levels of ApoB100 in each condition. FIG. 10B depicts the purity of EVs with respect to free proteins determined by dividing relative EV yield (the average of the ratios of CD9, CD63, and CD81) by levels of albumin in each condition. FIG. 10C depicts the relative EV recovery calculated for EV isolation from plasma. Simoa measurements for CD9, CD63, and CD81 were taken as a ratio relative to measurements of these proteins from diluted plasma and these three rations are then averaged to calculate recovery.

FIG. 11 depicts the percent recovery of different concentrations of purified ApoB-100 spike added to plasma measured using the ApoB-100 Simoa assay.

FIGS. 12A-12B depict that in-column PBS washes improve EV recovery. FIG. 12A depicts the levels of CD9, CD63, CD81, and albumin measured using Simoa after EV isolation of 1 mL plasma with SEC using 0, 1, 2, or 3 in-column 10 mL PBS washes. FIG. 12B depicts the percent recovery of EVs using average of ratios of CD9, CD63, and CD81 in SEC isolation relative to plasma.

FIGS. 13A-13B depict the analysis of markers in individual fractions of SEC and dual mode chromatography (DMC). FIG. 13A depicts the levels of CD9, CD63, CD81, ApoB-100 and albumin in fractions 7-14 for SEC with 10 mL Sepharose CL-6B column and DMC using a column with 2 mL Fractogel cation exchange bottom layer and 10 mL Sepharose CL-6B top layer. Simoa analysis of CD9, CD63, CD81, ApoB-100, and albumin were performed in different fractions after loading 1 mL plasma into a 10 mL Sepharose CL-6B SEC column or a DMC column (with 10 mL Sepharose CL-6B and 2 mL cation exchange resin). FIG. 13B depicts the concentrations of CD9, CD63, CD81, ApoB-100 and albumin in EV samples isolated from 1 mL plasma using SEC (fractions 7-10), DMC (fractions 9-12), or TMC columns (fractions 9-12). Error bars represent the standard deviation for four columns measured on different days with two technical replicates each.

FIGS. 14A-14C are related to an embodiment of a device configured to hold a twenty-four well chromatography plate. FIG. 14A schematically depicts a perspective view of a chromatography stand holder integrated with a liquid handling platform. FIG. 14B schematically depicts a perspective view of a chromatography plate holder including a twenty-four well chromatography plate. FIG. 14C schematically depicts an exploded view of a chromatography plate holder including a twenty-four well chromatography plate and a cap sized for the plate.

FIG. 15A-15C depict the analysis of markers in individual fractions of SEC. FIG. 15A depicts the levels of CD9, CD63 and albumin in fractions 5-10 for SEC in a 24-well chromatography plate with 7 or 8 mL Sepharose CL-6B resin. Simoa analysis of CD9, CD63 and albumin were performed in different fractions after loading 0.5 mL plasma into a 24-well chromatography palte with 7 mL Sepharose CL-6B SEC resin or 8 mL Sepharose CL-6B SEC resin. FIG. 15B depicts the levels of CD9, CD63, and albumin in fractions 5-10 for SEC with 9 mL Sepharose CL-6B column and 10 mL Sepharose-CL6B column. Simoa analysis of CD9, CD63 and albumin were performed in different fractions after loading 0.5 mL plasma into a 9 mL Sepharose CL-6B SEC column or a 10 mL Sepharose CL-6B SEC column. FIG. 15C depicts relative levels of EV yield and relative EV purity. Relative levels of EV yield were calculated by averaging relative levels of CD9 and CD63 for 24 well plates with 7 mL or 8 mL of Sepharose-CL6B resin to 9 mL Sepharose-CL6B column and 10 mL Sepharose-CL6B column. Relative EV purity was calculated by dividing EV yield by albumin levels for 24 well plates with 7 mL or 8 mL of Sepharose-CL6B resin to 9 mL Sepharose-CL6B column and 10 mL Sepharose-CL6B column.

### DETAILED DESCRIPTION

The present disclosure provides devices and methods for automated chromatography analysis of multiple samples in parallel.

SEC is theoretically suitable for running clinical samples as it is inexpensive and takes a short time to perform. The throughput of SEC, however, is limited. SEC columns are usually run one at a time, and although it's possible to run more than one column at the same time, this becomes challenging if done manually. To increase the throughput and reproducibility of SEC, disclosed herein is an automated stand for running eight columns in parallel.

Accordingly, described herein are novel tri-mode chromatography columns and methods for purification of extracellular vesicles (EVs) from circulating proteins and other contaminants present in a sample, e.g., a biological sample such as plasma.

Extracellular vesicles (EVs) are released by all cells into biofluids such as plasma. The separation of EVs from highly abundant free proteins and similarly-sized lipoproteins remains technically challenging. Previous solutions employed a digital ELISA assay based on Single Molecule Array (Simoa) technology for ApoB, the main protein component of lipoproteins. Combining this ApoB assay with previously developed Simoa assays for Albumin and three tetraspanin proteins found on EVs allows for measurement of the separation of EVs from both lipoproteins and free proteins.

The inventors developed improved methods for EV purification and isolation based on mixing several types of chromatography resins in a single column. Specifically, the inventors have successfully demonstrated, for the very first time, that by combining three different chromatography resins, e.g., a size exclusion chromatography resin, a cation exchange chromatography resin, and size-exclusion beads, in a single column, they were able to isolate EVs with high purity and significantly remove the major contaminating proteins, such as albumin and lipoproteins, from plasma samples. This represents a significant improvement over all existing EV purification methods.

The inventors have also demonstrated that the tri-mode chromatography column of this application confers additional advantages over the commonly used EV purification methods in that a small sample size, e.g., 1 mL, is sufficient for EV purification. In addition, the tri-mode chromatography column of the invention allows EV isolation in one single step, which is greatly advantageous for processing clinical samples for diagnostics, especially given that the chromatography column is also compatible with the automation device of the invention, as described herein.

Furthermore, the placement of the size exclusion beads in the bottom layer of the tri-mode chromatography column serves to “catch” residual free protein that was not fully separated by the size exclusion chromatography resin. Another advantage of the tri-mode chromatography column is that by mixing the cation exchange chromatography resin and the size exclusion beads in the bottom layer of the tri-mode chromatography column, the inventors were able to produce EVs of superior purity than the cation exchange chromatography resin or size exclusion beads alone in the bottom layer, which indicates that there is an advantage to having the mixture for the optimal performance of both individual resins.

Moreover, the tri-mode chromatography column of the invention is also particularly useful for EV biomarker discovery using proteomics. As demonstrated in the application, the inventors were able to detect almost 800 proteins from EVs isolated from only 1 mL of plasma samples using the tri-mode chromatography column in a single-step isolation protocol, further proving the advantage of the tri-mode chromatography column for deep proteomics analysis using a small sample volume.

### I. Definitions

In order that the present invention may be more readily understood, certain terms are first defined.

Unless otherwise defined herein, scientific and technical terms used in connection with the present invention shall have the meanings that are commonly understood by those of ordinary skill in the art. The meaning and scope of the terms should be clear, however, in the event of any latent ambiguity, definitions provided herein take precedent over any dictionary or extrinsic definition.

The use of the terms “a” and “an” and “the” and similar referents in the context of describing the invention (especially in the context of the following claims) are to be construed to cover both the singular and the plural (i.e., one or more), unless otherwise indicated herein or clearly contradicted by context.

The terms “comprising, “having,” “including,” and “containing” are to be construed as open-ended terms (i.e., meaning “including, but not limited to”) unless otherwise noted. Recitation of ranges of values herein are merely intended to serve as a shorthand method of referring individually to each separate value recited or falling within the range, unless otherwise indicated herein, and each separate value is incorporated into the specification as if it were individually recited.

The term “about” or “approximately” means within 5%, or more preferably within 1%, of a given value or range.

As used herein, the term “substantially” refers to the qualitative condition of exhibiting total or near-total extent or degree of a characteristic or property of interest. One of ordinary skill in the art will understand that biological and chemical phenomena rarely, if ever, go to completion and/or proceed to completeness or achieve or avoid an absolute result.

The term “substantially” may therefore be used in some embodiments herein to capture potential lack of completeness inherent in many biological and chemical phenomena.

As used herein, the term “extracellular vesicles (EVs)” refer to a class of membrane bound organelles secreted by various cell types. By “extracellular vesicle” as provided herein is meant a cell-derived vesicle having a membrane that surrounds and encloses a central internal space. Membranes of EVs can be composed of a lipid bi-layer having an external surface and an internal surface bounding an enclosed volume. Such membranes can have one or more types of cargo, such as proteins, embedded therein. EVs include all membrane-bound vesicles that have a cross-sectional diameter smaller than the cell from which they are secreted. EVs can have a longest dimension, such as a longest cross-sectional dimension, such as a cross-sectional diameter ranging from 10 nm to 1000 nm, such as 20 nm to 1000 nm, such as 30 nm to 1000 nm, such as 10 to 100 nm, such as 20 to 100 nm, such as 30 to 100 nm, such as 40 to 100 nm, such as 10 to 200 nm, such as 20 to 200 nm, such as 30 to 200 nm, such as 40 to 200 nm, such as 10 to 120 nm, such as 20 to 120 nm, such as 30 to 120 nm, such as 40 to 120 nm, such as 10 to 300 nm, such as 20 to 300 nm, such as 30 to 300 nm, such as 40 to 300 nm, such as 50 to 1000 nm, such as 500 to 2000 nm, such as 100 to 500 nm, such as 500 to 1000 nm and such as 40 nm to 500 nm, each range inclusive.

EVs are important for intercellular communications within the human body and involved in many pathophysiological conditions such as cancer or neurodegenerative disease. EVs are abundant in various patient biological samples, e.g., biological fluids, including but not limited to blood, plasma, serum, cerebrospinal fluid, urine, saliva, breast milk, synovial, amniotic, and lymph fluids. Membrane proteins can reflect the cellular environment the EV came from, for example a healthy or a tumor cell, or from a particular cell type, for example a specific breast cancer cell type. This genetic material can also hold clues to where the EV came from in the body, and how the EV may be interacting as a signaling messenger in the body.

The ability to detect EVs in various patient biological fluids has been shown to correlate well with disease progression, immune response, and toxicity; thus, the measurement and quantification of EVs could aid in disease diagnosis, and monitoring of treatment response. All disease states can have molecular signatures reflected in EVs. EVs can have molecular signatures reflected in, for example, but not limited to, cancer progression, cancer metastasis, melanoma, breast cancer, lung cancer, ovarian cancer, kidney cancer, glioblastoma, brain cancer, development of autoinflammatory disease such as Systemic lupus erythematosus, rheumatoid arthritis, multiple sclerosis, inflammatory bowel disease, neurodegenerative diseases such as Alzheimer's disease or Parkinson's disease, prion disease, transmissible spongiform encephalopathy, Creutzfeldt-Jakob disease, synucleinopathy, Dementia, multiple system atrophy, Huntington's disease, amyotrophic lateral sclerosis, leukemia, and more.

EVs as provided herein include exosomes. By “exosome” is meant a cell-derived vesicle composed of a membrane enclosing an internal space, wherein the vesicle is generated from a cell by fusion of the late endosome with the plasma membrane or by direct plasma membrane budding, and wherein the vesicle has a longest dimension, such as a longest cross-sectional dimension, such as a cross-sectional diameter, ranging for example, from 10 nm to 150 nm, such as 20 nm to 150 nm, such as 20 nm to 130 nm, such as 20 nm to 120 nm, such as 20 to 100 nm, such as 40 to 130 nm, such as 30 to 150 nm, such as 40 to 150 nm, or from 30 nm to 200 nm, such as 30 to 100 nm, such as 30 nm to 150 nm, such as 40 nm to 120 nm, such as 40 to 150 nm, such as 40 to 200 nm, such as 50 to 150 nm, such as 50 to 200 nm, such as 50 to 100 nm, or from 10 to 400 nm, such as 10 to 250 nm, such as 50 to 250 nm, such as 100 to 250 nm, such as 200 to 250 nm, such as 10 to 300 nm, such as 50 to 400 nm, such as 100 to 400 nm, such as 200 to 400 nm, each range inclusive. As used herein, “inclusive” refers to a provided range including each of the listed numbers. Unless noted otherwise herein, all provided ranges are inclusive.

An exosome is typically created intracellularly when a segment of the cell membrane spontaneously invaginates and is ultimately exocytosed. As used herein, exosomes can also include any shed membrane bound particle that is derived from either the plasma membrane or an internal membrane. Exosomes can also include cell-derived structures bounded by a lipid bilayer membrane arising from both herniated evagination (blebbing) separation and sealing of portions of the plasma membrane or from the export of any intracellular membrane-bounded vesicular structure containing various membrane-associated proteins, including surface-bound molecules derived from the host circulation that bind selectively to the exosomal proteins together with molecules contained in the exosome lumen, including but not limited to mRNAs, microRNAs or intracellular proteins. Blebs and blebbing are further described in Charras et al, Nature Reviews Molecular and Cell Biology, Vol. 9, No. 11, p. 730-736 (2008). Exosomes can also include membrane fragments.

The term “bead” means a small discrete particle that may be used to capture molecules, e.g., molecules of a certain size. Suitable beads include, but are not limited to, paramagnetic beads, plastic beads, ceramic beads, glass beads, polystyrene beads, methylstryene beads, acrylic polymer beads, carbon graphited beads, titanium dioxide beads, latex or cross-linked dextrans such as Sepharose® beads, cellulose beads, nylon beads, cross-linked micelles, and Teflon® beads. In some embodiments, spherical beads are used, but it is to be understood that non-spherical or irregularly-shaped beads may be used. In some embodiments, the bead is a “size-exclusion bead.”

As used herein, a size-exclusion beads is a bead that excludes molecules that are greater than a certain size from entering the core of the bead and molecules less than a certain size are captured by, or bind to, the core of the bead. In some embodiments, the beads exclude molecules that are the size of extracellular vesicles or greater. In some embodiments, the beads exclude molecules which are greater than the size of a target EV, e.g., greater than about 700 kDa. In some embodiments, the beads comprise an inactive bead exterior or shell. In some embodiments, the shell comprises pores. In some embodiments, the beads contain a ligand-activated core such as an octylamine ligand. In some embodiments, the size-exclusion beads comprise bind-elute resins. In some embodiments, the size-exclusion beads are Capto™ Core resin beads, e.g., Capto™ Core 700 resin beads.

As used herein, “subject” refers to any animal. In some embodiments, the subject is a human. Other animals that can be subjects include but are not limited to non-human primates (e.g., monkeys, gorillas, and chimpanzees), domesticated animals (e.g., horses, pigs, donkeys, goats, rabbits, sheep, cattle, yaks, alpacas, and llamas), and companion animals (e.g., cats, dogs, hamsters, guinea pigs, rats, mice, and birds.)

As used herein, “biological sample” refers to any biological sample obtained from or derived from a subject. In some embodiments, the biological sample contains EVs. In a preferred embodiment, the biological sample is a liquid biological sample.

The term “liquid sample” or “liquid biological sample,” as used herein, refers to a sample that is substantially in liquid form. In some embodiments, a liquid sample is a body fluid. Body fluids include, e.g., serum (including fresh or frozen), plasma (including fresh or frozen), peripheral blood mononuclear cells, whole blood (including fresh or frozen), cerebrospinal fluid (CSF), synovial fluid, urine, lymph, saliva, semen, sputum, mucous, feces, and vaginal fluid. In some embodiments, the biological sample is a plasma sample. In some embodiments, the biological sample is a CSF sample.

It should be noted that whenever a value or range of values of a parameter are recited, it is intended that values and ranges intermediate to the recited values are also intended to be part of this invention.

### II. Chromatography Devices

Conventional solutions for automated chromatography, for example, AKTA® machines, are capable of running only one sample at a time. After running one sample, the machine must be cleaned, a process that takes hours to properly avoid contamination of the next sample. However, the device discussed herein is capable of running at least eight sample in parallel before replacing the components for a new series of tests.

Another conventional chromatography solution is produced by Izon®. Izon® offers an automated analysis of a single sample split into many fractions. But there is not a solution for running multiple samples in parallel as discussed herein. Analysis of multiple samples in parallel greatly reduces overall testing time compared to analysis of a single sample at a time.

The disclosed device also offers advantageous compared to manual loading of samples. Pipetting and running a 1 mL sample into a chromatography column requires about fifteen minutes of time. However, the disclosed device automates the introduction of the liquid sample, performing a test on at least eight samples in the same time it would take to manually run one sample.

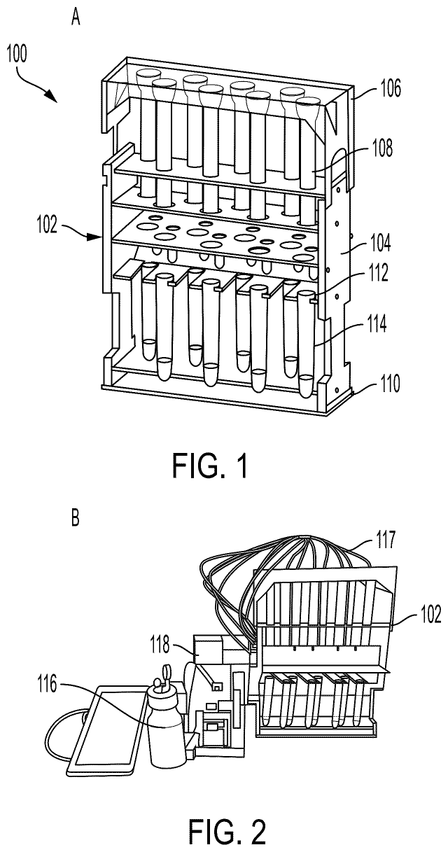

FIG. 1 schematically depicts a perspective view of a chromatography stand 100. In some embodiments, the device 100 includes a stand 102 including a frame 104, an upper portion 106 attached to the frame 104, and a bottom portion 110 connected to the frame 104. The device 100 may further include a pump 116 and a computing device 118, which are discussed in further detail with regards to FIG. 2.

The frame 104 may be composed of any suitable material sufficiently strong enough to securely contain at least eight tubes and columns. By way of non-limiting example, such materials may include metals, porous polymeric materials, silica, carbon based solids, or any other suitable material known in the art. The frame 104 is configured to withstand forces from liquid flow introduced into the upper portion 106 without the stand 102 knocking over or otherwsise shifting position. In some embodiments, the frame 104 is substantially symmetrical such that the upper portion 106 and bottom portion 110 are in alignment with one another. In other embodiments, the frame 104 may be assymetrical such that the upper portion 106 and the bottom portion 110 may not be in alignment or may not be wholly situated above one another.

The upper portion 106 includes at least one column 108. In some embodiments, the upper portion 106 includes eight columns 108. Each column 108 may be separate from one another. Alternatively, each column 108 may be a part of a well plate, for example, a twenty-four or ninety-six well plate, where each well on the plate is considered as a chromatography column. The columns 108 may be composed of any material known to be suitable in the art. Each column 108 may contain a resin reactive to a liquid sample. Useful resins are discussed in further detail below. In some embodiments, the column includes a size exclusion chromatograpy resin, a cation-exchange resin, and/or size exclusion beads capable of capturing molecules smaller than about 700 kDa.

The bottom portion 110 includes at least one opening 112. In some embodiments, the bottom portion 110 includes at least one row of openings 112. In embodiments including multiple rows, the rows may be in parallel or in a staggered formation. In some embodiments, each row contains eight openings 112.

Each opening may be configured to house a well or tube 114, for example an Eppendorf tube. Each tube may be sized to receive any where from half a milliliter biological sample, for example blood, plasma, or waste that passed through the assay in a column 108, to fifty milliliters of sample. For example, each tube or well may be sized to receive 0.5 mL, 1.0 mL, 1.5 mL, 2.0 mL, 5.0 mL, 10 mL, 20 mL, 25 mL, or 50 ml of sample, or any amount therebetween. In some embodiments, the bottom portion 110 may be positioned such that a row of openings 112 is positioned to receive the liquid sample or waste from the upper portion 106.

In some embodiments, the bottom portion 110 is configured to house at least eight tubes or wells 114. In some embodiments, the bottom portion 110 is configured to house at least twenty-four tubes or wells 114. In some embodiments, the bottom portion 110 is configured to house at least ninety-six tubes or wells 114. In embodiments including ninety-six tubes or wells 114, the rows may be staggered formation or in a grid pattern.

FIG. 2 depicts a photograph of a chromatography stand 100 connected to a computing device 118 and a pump 116. The pump 116 is configured to deliver the biological sample to the stand 102, or the resins described herein. In some embodiments, the pump 116 is a syringe pump. The pump 116 may be loaded with a biological sample to be analyzed with chromatography, such as blood or plasma containing extracellular vesicles. The pump 116 may be any conventional pump known to one of skill in the art, for example a robotic liquid handler.

The pump 116 is in fluid communication with the upper portion 106. More specifically, pump 116 may include one or more cannulas 117. An individual canula 117 may be in fluid communication with a column 108 within the upper portion 106. For example, the pump 116 may include eight cannulas 117, each cannula 117 in fluid communication with one of eight columns 108 of the upper portion 106 such that the liquid sample is introduced into each column 108.

Operation of the pump 116 is controlled by a computer device 118 configured to control the flow of the biological sample from the pump 116. The computing device 118 may be any conventional computing device capable of controlling operation of a pump, for example a Raspberry Pi®. A user may program the computing device 118 to operate the pump at a desried flow rate for a desired time.

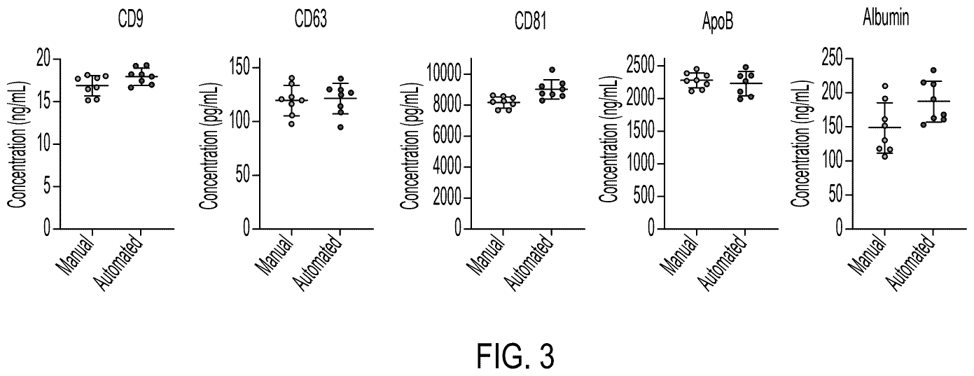

FIG. 3 depicts Simoa comparisons of various assays when chromatography was performed on samples manually and automatically. Using a pump run by a Raspberry Pi, a liquid sample of plasma was dispensed to all columns in parallel. Reproducibility of the device was tested by comparing Simoa results for eight columns run by automation compared to eight columns run manually. Simoa comparison of CD9, CD63, CD81, ApoB100 and Albumin was conducted when SEC was performed on plasma using eight columns run manually and in the automated device. The results of both tests were appreciably similar, establishing that an automated device produces equally useful results as compared to manual testing of samples.

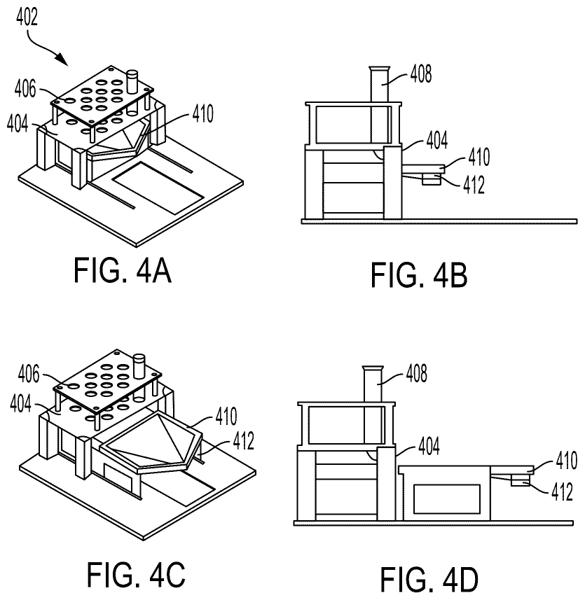

FIGS. 4A-4D are related to an embodiment of a chromatography stand 402 including a slideable bottom portion 410. In some embodiments, the bottom portion 410 is slideably connected to the frame 404. The bottom portion 410 may include a waste collection element 412 that collects waste that passes through the columns 408. In some embodiments, the tubes or wells form at least a portion of the waste collection element 412.

In some embodiments, stand 402 may include twenty-four or ninety-six wells. In emboidments including ninety-six wells, the columns 408 may be arranged in a grid pattern instead of in a row.

In some embodiments, upper portion 406 of the stand 402 may include more than eisght columns 408. For example, upper portion 406 may include twelve columns 408.

FIG. 4A schematically depicts a perspective view of the chromatography stand 402 wherein the slideable bottom portion 410 is positioned under an upper portion 406. FIG. 4B schematically depicts a side view of the chromatography stand 402 wherein the slideable bottom portion 410 is positioned under an upper portion 406. In this position, the waste collection element 412 may collect waste that drips from the columns 408.

FIG. 4C schematically depicts a perspective view of a chromatography stand including a slideable bottom portion slid out from under an upper portion. FIG. 4D schematically depicts a side view of a chromatography stand including a slideable bottom portion slid out from under an upper portion. In this position, collected fluid may be drained or emptied from the waste collection element 412.

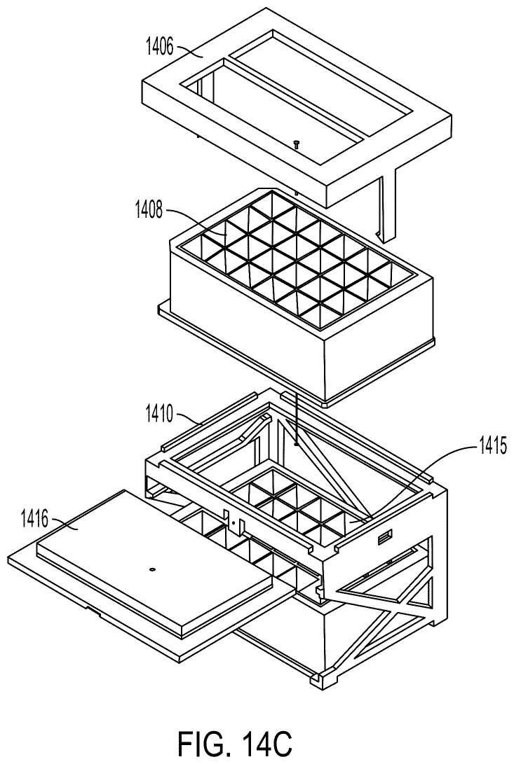

FIGS. 14A-14C are related to an embodiment of a chromatography stand holder 1400 configured to hold a twenty-four well chromatography plate 1414. The plate 1414 may be housed within a bottom portion 1410 of the device 1400. An upper portion 1406 may be positioned above the bottom portion 1410. The upper portion may house one or more columns 1408.

In some embodiments, plate 1414 may include twenty-four wells 1415. The wells 1415, and correspondng columns 1408, may be arranged in a grid.

FIG. 14A schematically depicts a perspective view of a chromatography stand holder 1400 integrated with a liquid handling platform 1430. The liquid handling handling platform may accommodate multiple chromatography stand holders 1400.

FIG. 14B schematically depicts a perspective view of a chromatography plate holder 1400 including a twenty-four well chromatography plate 1414. In some embodiments, the bottom portion 1410 may be positioned such that each well 1415 is positioned to receive a liquid sample or waste from the columns 1408 of the upper portion 1406. FIG. 14C schematically depicts an exploded view of a chromatography plate holder 1400 including a twenty-four well chromatography plate 1414 and a cap 1416 sized for the plate 1414. The cap 1416 may be placed onto the plate 1414 to stop the flow of liquid into the wells 1415. The cap 1416 may be removed to let the flow continue.

### III. Methods of Automated Chromatography

Methods of automating chromatography of multiple samples in parallel are disclosed herein. In some embodiments, the method includes providing a device as described in detail above including a stand, a pump, and a computing device. The stand includes a frame, an upper portion attached to the frame, and a bottom portion connected to the frame. In some embodiments, the upper portion includes a column containing a resin and configured to receive a biological sample. In some embodiments, the bottom portion comprising a row of openings, each opening configured to house a tube or well. In some embodiments, the bottom portion may be positioned such that the row of openings is positioned to receive the biological sample from the upper portion.

The method further includes selecting a sample for chromatography testing, for example blood or plasma. The disclosed method is particularly useful for the analysis of plasma. As described herein, the assays employed in the disclosed device advantageously separates EV from the proteins in plasma.

The method further inclues initiating a flow of the biological sample into the stand. In some embodiments, the flow is provided by a pump as discussed in further detail above. The pump may connected to columns within the stand by one or more cannulas positioned above or within the columns.

Operation of the pump is controlled by a computing device, for example a Rasberry Pi®. A user may program the computing device to control a flow rate of the sample and a duration of the flow. For example, the computing device may be programmed to to disperse an entire eight mL sample over fifteen minutes into eight different columns, testing one mL of sample in each column.

In some embodiments, waste from the columns may be collected within wells and/or a waste collection element of the bottom portion of the stand. In some embodiments, the bottom portion may be slid out from the device to drain the wells and/or waste collection element. Waste may be drained after the computing device concludes flow of the liquid sample, or the flow may be manually paused to manipulate the stand to drain waste.

After the flow of sample is concluded, a user may collect fractions containing extracellular vesicles from the column, thereby purifying the extracellular vesicles. The user may then remove the columns and tubes/wells from the stand for analysis and cleaning. New columns and tubes/wells may be inserted into the stand to run another test. Similarly, the pump may be detached from the computing device and a new pump with a new sample may be attached.

### IV. Chromatography Columns and Methods for Purification of Extracellular Vesicles

The present disclosure provides a novel tri-mode chromatography column for purification of extracellular vesicles (EVs) from circulating proteins and other contaminants present in a sample, e.g., a biological sample such as plasma. Methods for purification of EVs using the tri-mode chromatography column are also described herein.

In particular, the inventors have successfully demonstrated, for the very first time, that by combining three different chromatography resins, e.g., a size exclusion chromatography resin, a cation exchange chromatography resin, and size-exclusion beads, in a single column, they were able to isolate EVs with high purity and significantly remove the major contaminating proteins, such as albumin and lipoproteins, from plasma samples. This represents a significant improvement over all existing EV purification methods.

The inventors have also demonstrated that the tri-mode chromatography column of this application confers additional advantages over the commonly used EV purification methods in that a small sample size, e.g., 1 mL, is sufficient for EV purification. In addition, the tri-mode chromatography column of the invention allows EV isolation in one single step, which is greatly advantageous for processing clinical samples for diagnostics, especially given that the chromatography column is also compatible with the automation device of the invention, as described above.

Furthermore, a crucial feature of the tri-mode chromatography column is that the size exclusion beads are placed in the bottom layer of the column and “catches” residual free protein that was not fully separated by the size exclusion chromatography resin. Another advantage of the tri-mode chromatography column is that by mixing the cation exchange chromatography resin and the size exclusion beads in the bottom layer of the tri-mode chromatography column, the inventors were able to produce EVs of superior purity than the cation exchange chromatography resin or size exclusion beads alone in the bottom layer, which indicates that there is an advantage to having the mixture for the optimal performance of both individual resins.

Moreover, the tri-mode chromatography column of the invention is also particularly useful for EV biomarker discovery using proteomics. As demonstrated in the application, the inventors were able to detect almost 800 proteins from EVs isolated from only 1 mL of plasma samples using the tri-mode chromatography column in a single-step isolation protocol, further proving the advantage of the tri-mode chromatography column for deep proteomics analysis using a small sample volume.

Accordingly, the present invention provides a chromatography column for purifying extracellular vesicles from a biological sample comprising: (a) a size exclusion chromatography resin, (b) a cation exchange chromatography resin; and (c) size-exclusion beads capable of capturing molecules smaller than about 700 kDa; wherein the size exclusion chromatography resin is placed at a top layer of the column; and wherein the cation exchange chromatography resin and the size-exclusion beads are mixed and the mixture is placed at a bottom layer of the column.

In another aspect, the present invention provides a method of purifying extracellular vesicles from a biological sample, the method comprising: (a) providing a chromatography column of the invention, (b) introducing a biological sample comprising extracellular vesicles into the column, and (c) collecting fractions containing extracellular vesicles from the column, thereby purifying the extracellular vesicles.

In some embodiments, the size exclusion chromatography resin comprise a Sepharose™ resin, e.g., a Sepharose™ cross-linked resin. In size exclusion chromatography, a porous stationary phase is utilized to sort macromolecules and particulate matters according to their size. Components in a sample with small hydrodynamic radii are able to pass through the pores, thus resulting in late elution. Components with large hydrodynamic radii, including EVs, are excluded from entering the pores. In some embodiments, the size exclusion chromatography resin comprises a Sepharose™ CL-6B resin.

In some embodiments, the cation exchange chromatography resin comprises a stationary phase comprising a functional group selected from the group consisting of sulpfhydryl, sulfonate, sulfate, carboxymethyl, sulfoethyl, sulfopropyl, phosphate and sulfonate. In some embodiments, the cation exchange chromatography resin is Fractogel® EMD-SO₃⁻ resin.

In some embodiments, the chromatography column further comprises size-exclusion beads. Size-exclusion beads can “trap” contaminants within the bead, thus enabling purification of extracellular vesicles from a sample, e.g., a liquid biological sample.

In some embodiments, the beads exclude molecules which are greater than the size of a target EV, e.g., greater than about 400, 500, 600, or 700 kDa. In some embodiments, the beads comprise an inactive bead exterior or shell. The exterior of the bead can comprise pores which allow molecules less than a certain size to pass through the exterior shell and be trapped in the core of the bead. In some embodiments, the shell comprises pores. In some embodiments, the beads contain a ligand-activated core such as a multimodal ligand, e.g., an octylamine ligand.

In some embodiments, the size-exclusion beads comprise a bind-elute resin. In some embodiments, the size-exclusion beads are Capto™ Core bind-elute beads. In some embodiments, the size-exclusion beads are Capto™ Core 700 bind-elute beads. Capto™ Core 700 chromatography resin (GE Healthcare Biosciences AB) comprise octylamine ligands within Capto™ Core 700 ‘beads’, and are designed to have both hydrophobic and positively charged properties that can trap molecules under 700 kDa. Since extracellular vesicles exceed 700 kDa, and since the bead exterior is inactive, Capto Core 700 permits purification of extracellular vesicles by size exclusion. With standard gel filtration (size exclusion chromatography), molecules of smaller size spend more time penetrating pores of the stationary phase, and therefore exhibit higher retention (slower elusion) relative to larger molecules. In contrast, the ligand-activated pores of Capto™ Core 700 have electrostatic and hydrophobic interactions that “capture” molecules under 700 kDa.

The size-exclusion beads can be suspended in a buffer to create a slurry. In some embodiments, the size-exclusion beads can be suspended in an equal volume of buffer to create a 50% slurry, or in any volume buffer effective to produce a slurry with an effective amount of size-exclusion beads to purify EVs from a sample. For example, the slurry can be a 20%, 30%, 40%, 50%, 60%, 70%, 80%, or 90% slurry. Any effective buffer can be used to produce the slurry. In some embodiments, PBS buffer is used.

In some embodiments, the chromatography column is a 10 mL, 12 mL, 15 mL, 20 mL, or 25 ml volume column.

In some embodiments, the top layer of the column comprises about 5 mL, about 6 mL, about 7 mL, about 8 mL, about 9 mL, or about 10 mL of the size exclusion chromatography resin.

In some embodiments, the top layer of the column comprises about 10 mL of the size exclusion chromatography resin.

In some embodiments, the bottom layer of the column comprises about 1 mL, about 2 mL, or about 4 mL of the mixture of the cation exchange chromatography resin and the size-exclusion beads. In some embodiments, the bottom layer of the column comprises about 2 mL of the mixture of the cation exchange chromatography resin and the size-exclusion beads.

In some embodiments, the cation exchange chromatography resin and the size-exclusion beads are mixed at a ratio of about 1:4, about 1:2, about 2:1, or about 4:1. In some embodiments, wherein the cation exchange chromatography resin and the size-exclusion beads are mixed at a ratio of about 2:1.

The tri-mode chromatography column can be prepared by first washing the resins prior to addition to the column. In some embodiments, the resins are washed in buffer, e.g., PBS, prior to preparation of the column. The resin can be washed multiple times prior to preparation of the column.

Once the resins are washed, they can be added to a suitable column, e.g., a 15 mL or 20 mL column. For example, the Fractogel® EMD-SO₃⁻ resin and the Capto™ Core 700 beads can be mixed at a ratio of about 2:1 by volume, and a 2 mL of the mixture can be added to the bottom layer of the column, followed by the addition of 10 mL Sepharose™ CL-6B resin to the top layer of the column.

The column may be washed prior to introducing the sample. In some embodiments, the column is washed with PBS.

Fractions containing EVs can be collected from the tri-mode column, thereby purifying the EVs from the biological sample. For example, fractions 9-12 can be collected for the tri-mode chromatography column.

While not necessary, following purification of EVs using any of the methods of the present disclosure, the purified extracellular vesicles can be further purified by any means known in the art. In addition, the columns for purification of EVs as described herein can be combined with each other, and with other EV purification methods known in the art. For example, in some embodiments, cation exchange chromatography, size exclusion chromatography, such as gel permeation columns, centrifugation or density gradient centrifugation, and filtration methods can be used in combination with the methods of the disclosure. As another example, the EV purification methods of the disclosure can be used differential centrifugation, anion exchange and/or gel permeation chromatography, sucrose density gradients, organelle electrophoresis, magnetic activated cell sorting (MACS), or with a nanomembrane ultrafiltration concentrator.

In some embodiments, the biological sample is obtained from a subject. In certain embodiments, the biological sample is a liquid biological sample. One skilled in the art will recognize that a biological sample can be, but is not limited to, the following bodily fluids: peripheral blood, plasma, serum, cerebrospinal fluid (CSF), ascites, sputum, saliva, bone marrow, synovial fluid, aqueous humor, amniotic fluid, cerumen, breast milk, broncheoalveolar lavage fluid, semen (including prostatic fluid), Cowper's fluid or pre-ejaculatory fluid, female ejaculate, sweat, fecal matter, hair, tears, cyst fluid, pleural and peritoneal fluid, pericardial fluid, lymph, chyme, chyle, bile, interstitial fluid, menses, pus, sebum, vomit, vaginal secretions, mucosal secretion, stool water, pancreatic juice, lavage fluids from sinus cavities, bronchopulmonary aspirates or other lavage fluids. A biological sample may also include the blastocyl cavity, umbilical cord blood, or maternal circulation that may be of fetal or maternal origin. The biological sample may also be a tissue sample or biopsy, from which EVs may be obtained. In one embodiment, the biological sample is a plasma sample. In another embodiment, the biological sample is a CSF sample.

### EXAMPLES

### Example 1: Isolation of Extracellular Vesiles by Removal of Both Free Proteins and Lipoproteins

This example describes improvevd EV isolation methods that can separate EVs from lipoproteins and free proteins at levels of purity beyond those of previously described methods. It was envisioned that these methods enable applications such as EV proteomics for biomarker discovery from human biofluids, where high EV purity is crucial.

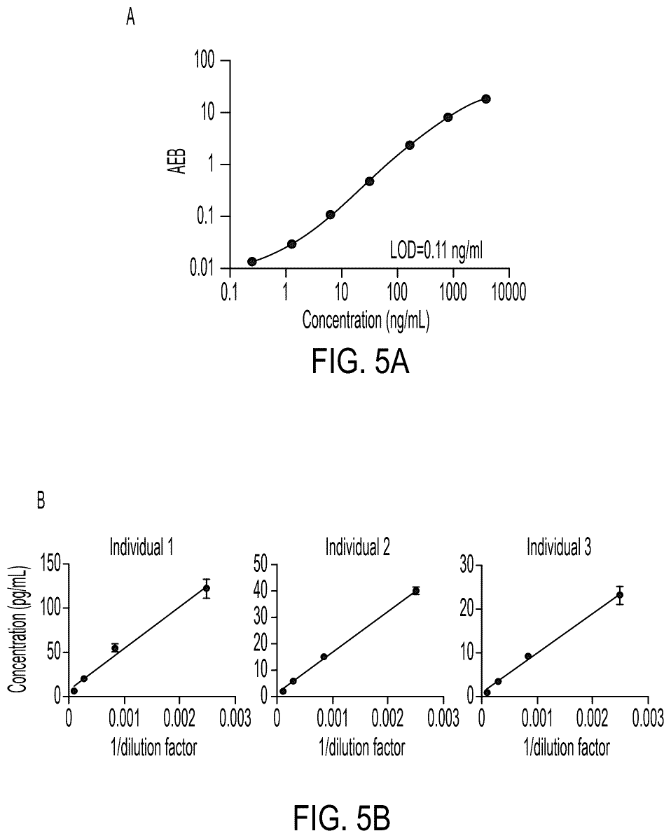

To measure lipoproteins, a Simoa assay against ApoB100, the protein component of lipoproteins such as LDL and VLDL, was first developed. After testing a variety of capture and detector antibodies, the best antibody pair was validated with dilution linearity experiments in three individual plasma samples (FIGS. 5A-5B). Spike and recovery experiments were also performed using recombinant protein standard (FIG. 11). This Simoa assay for ApoB100 was then combined with the previously developed CD9, CD63, CD81, and albumin assay to measure EVs, free proteins and lipoproteins on the same platform.

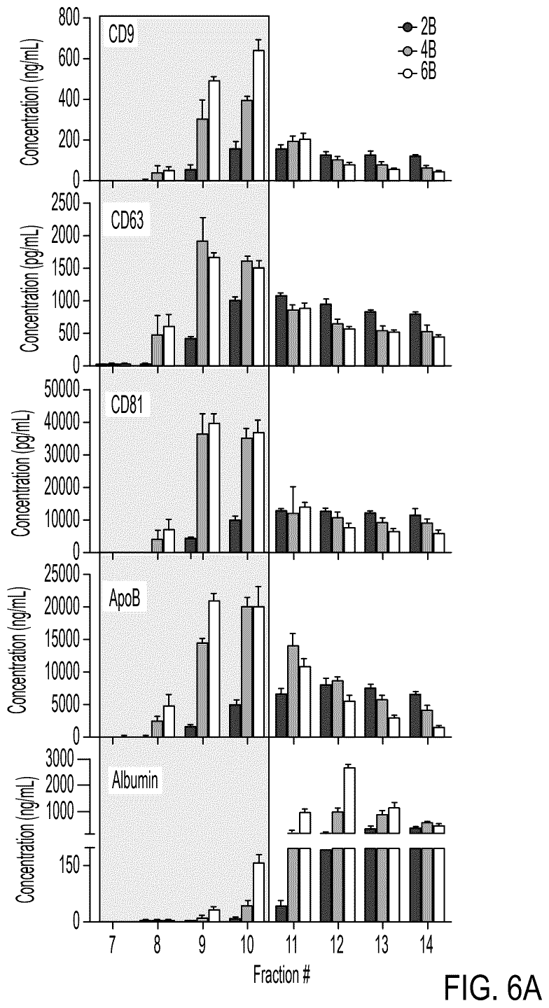

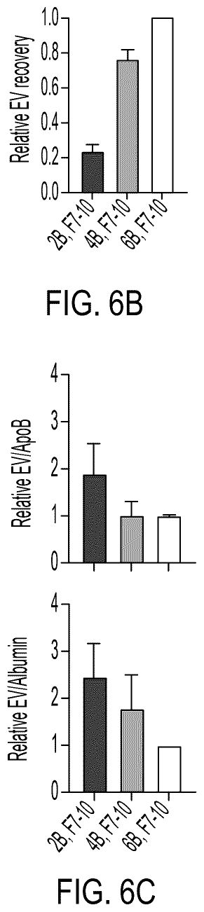

It was first investigated whether EVs can be separated from ApoB100-containing lipoproteins based on size. Human plasma samples were separated based on size using 10 mL size exclusion chromatography (SEC) columns using resins (Sepharose CL-2B, CL-4B, CL-6B) with three different pore sizes. 0.5 mL fractions were collected after performing SEC and used Simoa to measure CD9, CD63, CD81, albumin, and ApoB100 in each fraction (FIG. 6A). By averaging the ratios of each of the tetraspanin levels between conditions, the relative EV yields between different EV isolation methods were quantified (FIG. 6B). It was found that although the ratio of EVs relative to ApoB100 was higher in Sepharose CL-2B (FIG. 6C), this was at the expense of EV yield relative to the other two resins (FIG. 6B).

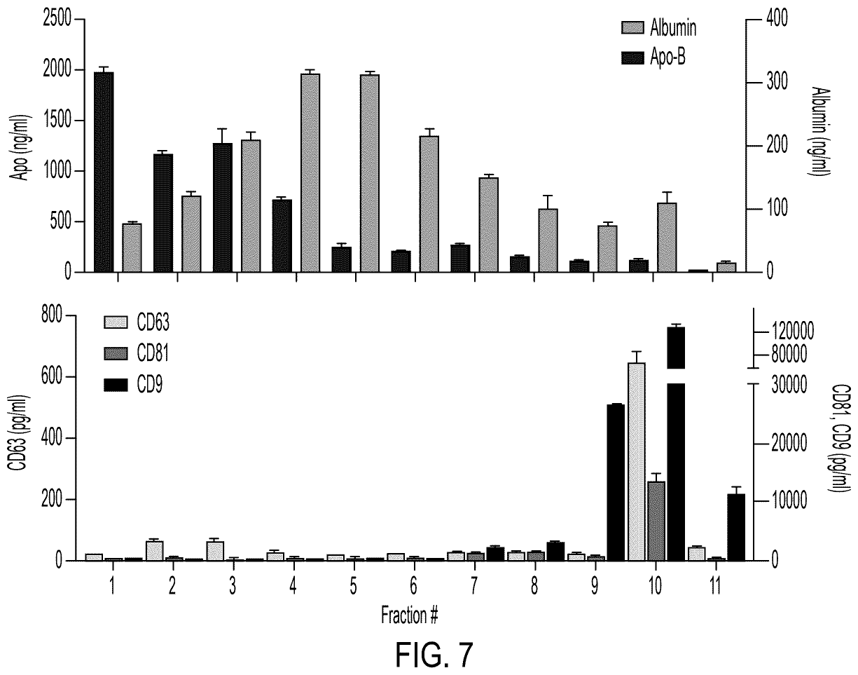

Since separating EVs from lipoproteins based on size alone is not possible, separation of EVs from lipoproteins based on other properties were explored. Separation of EVs from lipoproteins and albumin based on density using density gradient ultracentrifugation was first investigated. 1 mL of plasma was loaded on an iodixanol gradient and ultracentrifugation was performed for 16 hours. 1 mL fractions were collected and Simoa was used to measure tetraspanins, albumin and ApoB100 in each fraction. It was found that EVs could be readily separated from ApoB100-containing lipoproteins and albumin (FIG. 7). However, as density gradient ultracentrifugation is time-consuming and low throughput, other EV isolation methods that would be more suitable for diagnostic applications were explored.

SEC is theoretically suitable for running clinical samples as it is inexpensive and takes a short time to perform. The throughput of SEC, however, is limited. SEC columns are usually run one at a time, and although it's possible to run more than one column at the same time, this becomes challenging if done manually. To increase the throughput and reproducibility of SEC, a semi-automated stand for running eight columns in parallel was built. Using a pump run by a Raspberry Pi, liquid was dispensed to all columns in parallel (FIGS. 1-2). The reproducibility of this device was tested by comparing Simoa results for eight columns run by the device compare to eight columns run manually (FIG. 3).

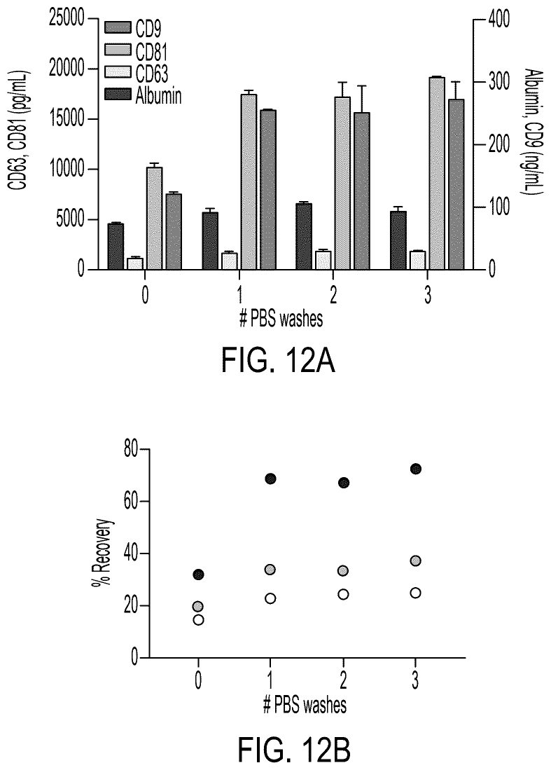

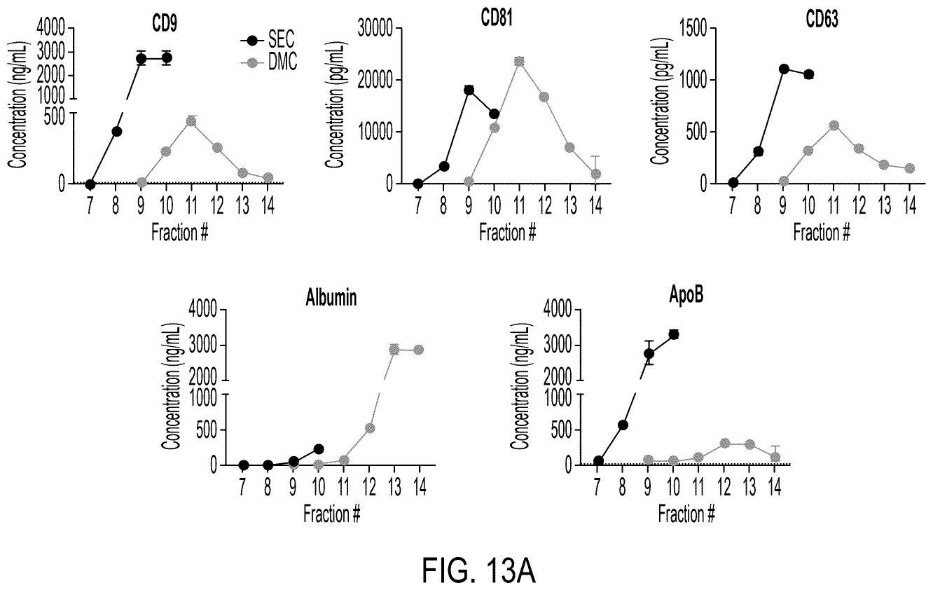

Next, it was considered whether SEC could be modified to maximize EV yield while removing both free proteins and lipoproteins. First, the absolute recovery of EVs by SEC from Sepharose CL-6B, the resin with highest EV yield, was investigated (FIG. 6B). The advantage of Simoa's high dynamic range and specificity to measure tetraspanin levels in diluted plasma was explored and compared with these levels after EV purification from the same batch of plasma by SEC. Various parameters were evaluated. It was found that performing at least one PBS washes in-column, as opposed to just washing the resin in bulk before making the column, increased the EV yield significantly (FIG. 12A). One potential reason for this could be that in-column washes are more effective at removing the ethanol in which the resin is supplied. After performing an in-column wash, >50% EV yield was achieved using SEC with Sepharose CL-6B (FIG. 12B), as measured by comparing tetraspanin levels in SEC fractions 7-10 relative to diluted plasma.

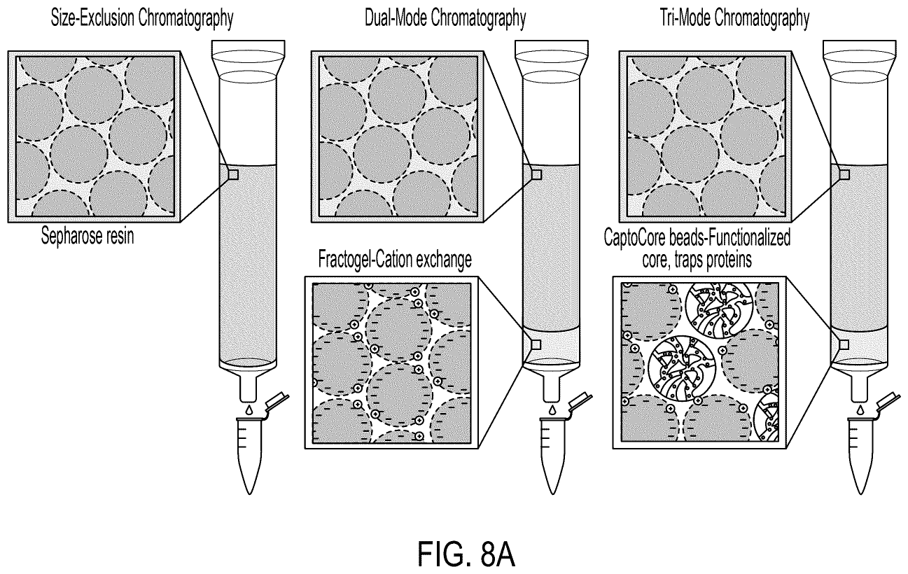

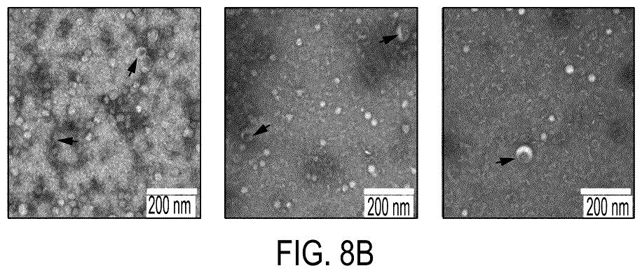

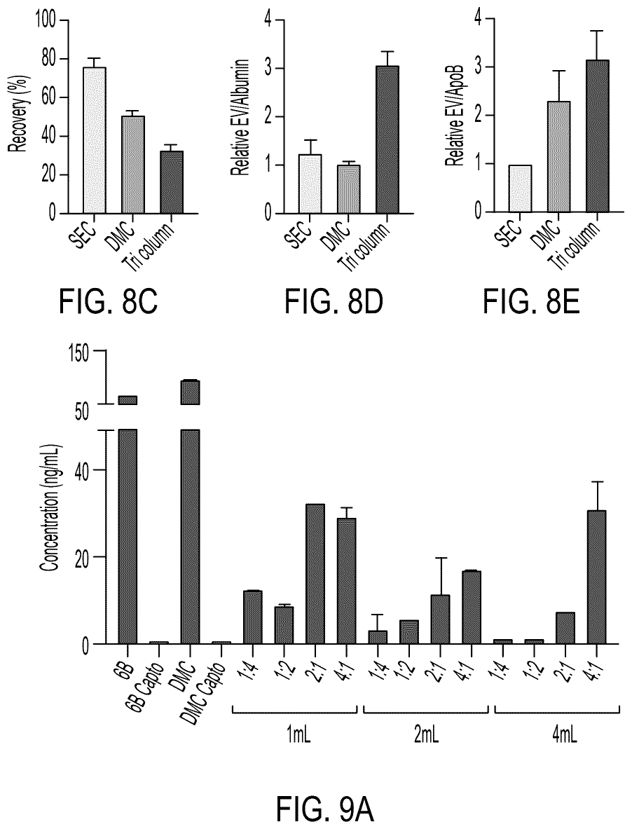

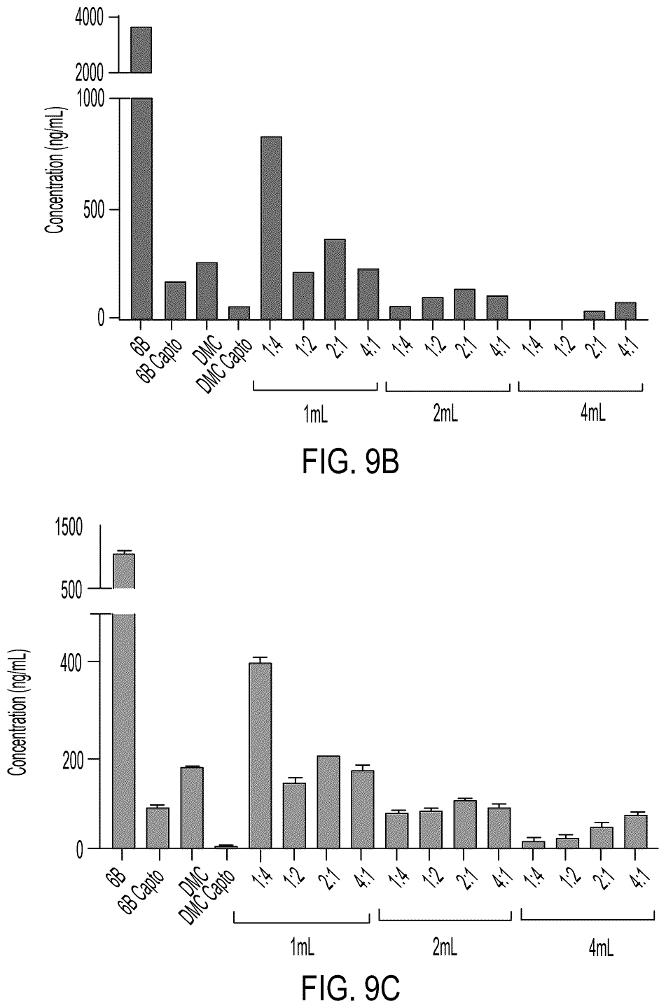

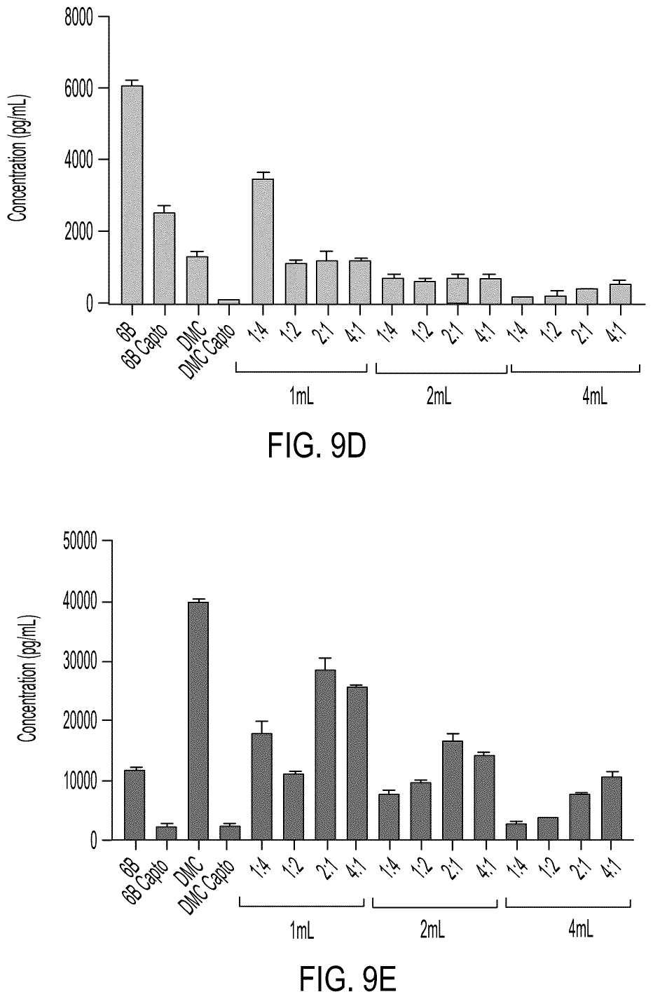

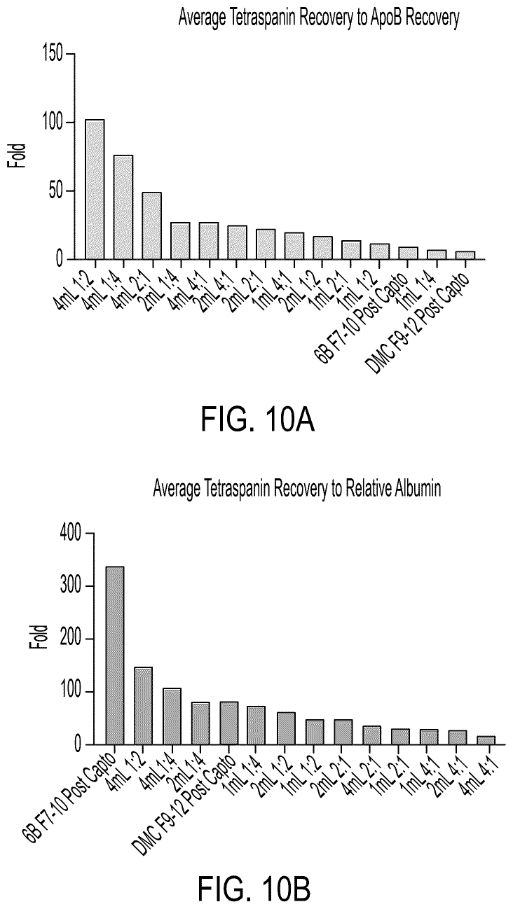

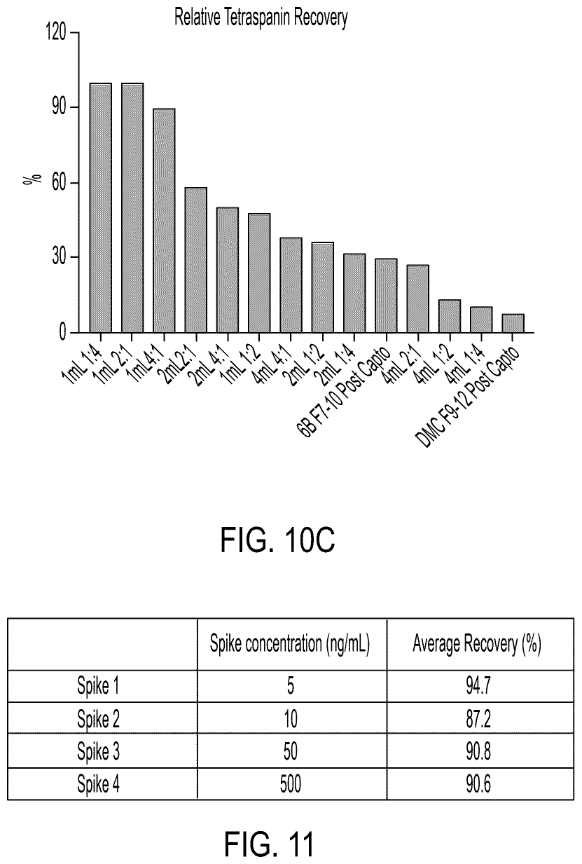

ApoB100 is positively charged, and EVs are generally negatively charged. The differential charge was explored to separate EVs from lipoproteins. It has previously been demonstrated that dual-mode chromatography (DMC) columns with a bottom layer of cation-exchange resin below Sepharose CL-4B can be used to isolate EVs. Since Sepharose CL-6B yields more EVs than Sepharose CL-4B (FIG. 6B), DMC columns were constructed with a 2 mL cation exchange resin bottom layer and a top layer of 10 mL Sepharose CL-6B. Inspired by the DMC approach of combining different resins in the same column, a new type of column, tri-mode chromatography (TMC), was also developed, where Capto Core 700 was added to the bottom layer of cation exchange resin. Capto Core 700 is a mixed-mode chromatography resin that contains porous beads with an inner core layer functionalized with octylamine groups that bind and trap proteins. Thus, it was reasoned that having this resin in the bottom layer would “catch” free proteins that co-isolate with EVs during SEC (FIG. 8A). After trying different ratios of resins, a two-to-one ratio of cation exchange to mixed-mode resin in the 2 mL bottom layer was selected (FIGS. 9A-9E, and FIGS. 10A-10C).

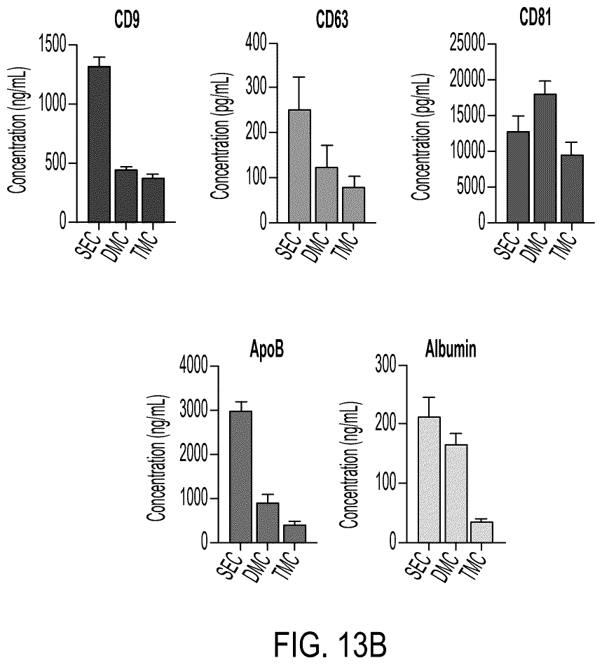

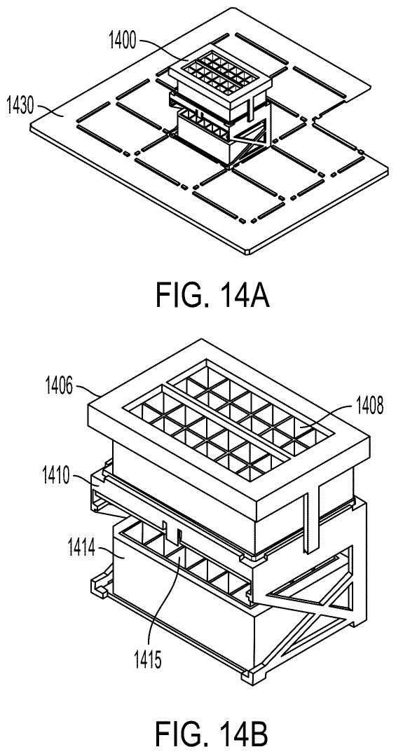

Next, EV isolation from plasma using SEC, DMC, and TMC columns were compared. First, electron microscopy was used to image EVs from each column and it was found that TMC led to EVs of the highest purity (FIG. 8B). Next, Simoa was used to quantify the relative levels of EVs, lipoproteins, and free proteins using SEC, DMC, and TMC columns. Fractions 9-12 (instead of fractions 7-10 as for SEC) were collected for DMC and TMC to account for the extra 2 mL of resin in the column (FIG. 13A). It was found that DMC and TMC columns significantly depleted ApoB 100, but also led to some loss in EV yield and, in particular, CD9, compared to SEC columns (FIG. 13B). Calculating the relative yields of each tetraspanin and averaging the three tetraspanin ratios to calculate EV yield, it was found that DMC and TMC columns had a lower EV yield than SEC but significantly higher ApoB/EV ratios. Although DMC columns had higher ratios of EVs to ApoB compared to SEC, the levels of EVs to albumin remained the same. The TMC column, on the other hand, had a higher ratio of both EVs to ApoB and EVs to albumin compared to the SEC column (FIGS. 8C-8E).

To assess the utility of highly pure EVs isolated with TMC, mass spectrometry-based proteomic analysis was performed. Performing mass spectrometry on EVs from plasma is challenging since levels of both free proteins and lipoproteins are several orders of magnitude higher than those of EV proteins. Using TMC, the inventors were able to detect 789 proteins from EVs isolated from only 1 ml of plasma. These results demonstrate the advantage of using TMC for deep proteomics analysis using a small sample volume.

### Dicussion

In this work, the inventors developed methods to separate EVs away from lipoproteins and free proteins in plasma based on our ability to measure proteins associated with these different components. First, a Simoa assay for ApoB100 as developed and validated. This assay was combined with previously developed Simoa assays for the tetraspanins CD9, CD63, and CD81, as well as albumin. With these assays in place, the inventors could quantify EVs, free proteins, and lipoproteins from the same sample on one experimental platform. Using this approach, different ways of separating EVs from lipoproteins were assessed with the aim of developing improved EV isolation methods.

Plasma contains several types of lipoproteins with varying protein and lipid compositions. Although there is not one protein present on all lipoproteins, ApoB100 was chosen to be measured as it is the most abundant protein component of several lipoproteins. ApoB100 is present on lipoproteins such as LDL and VLDL, and these particles overlap in size with the size range of EVs. Previous reports have suggested that SEC may be able to separate EVs from lipoproteins, and this possibility was assessed using this platform for SEC resins with three different pore sizes. Previously, the tetraspanin and albumin Simoa assays were used to directly compare EV yield and free protein contamination for different SEC resins. Here, a similar framework was used to compare EV yield and lipoprotein contamination. Although some separation of ApoB100 from the tetraspanins is possible by SEC, and this separation improves by using resins with larger pore sizes, this comes at the expense of EV yield. The Simoa assays were also used to evaluate density gradient centrifugation (DGC) and showed that this technique enable good separation of tetraspanins from ApoB100 and albumin. However, since DGC requires an ultracentrifuge, is low throughput, and time-intensive, it is not suitable for clinical samples.

Novel methods for separating EVs from lipoproteins were developed. A previous study described dual-mode chromatography (DMC) columns that deplete lipoproteins by combining SEC using Sepharose CL-4B with a second bottom layer of cation-exchange resin. DMC was modified to include the higher yield Sepharose CL-6B resin and demonstrated depletion of most of the ApoB100, although at the cost of some EV depletion. To improve the ratio of EVs to ApoB and albumin, a new method was developed that combines a top layer of Sepharose CL-6B with a bottom layer of both cation-exchange resin and a multimodal chromatography resin called Capto Core 700. These “TriModal mixed mode Chromatography,” or TMC columns, produced EVs of superior purity relative to both albumin and lipoproteins.

This example presents a framework for quantitatively comparing EV isolation methods. There is not a single optimal way to isolate EVs because the isolation method must be matched to the application, therefore, it is crucial to have effective ways of comparing both the yield and purity of different isolation methods. TMC columns were developed for applications where EVs of very high purity are needed and these columns were optimized for EV isolation from plasma. These columns would be particularly useful for EV biomarker discovery using proteomics, where EV contamination with lipoproteins and free proteins such as albumin prevents deep protein coverage. Using TMC columns, the inventors were able to measure the plasma EV proteome using an easy, single-step isolation protocol. By also building an automated device for running columns in parallel, the inventors demonstrate a path towards using column-based methods for clinical samples. Thus, the methods will enable the potential of EV proteomic profiling in molecular diagnostics.

### Methods and Materials

### Human Samples:

Pooled human plasma (collected in K2 EDTA tubes) was ordered from BioIVT. Plasma was thawed at room temperature and centrifuged at 2000×g for 10 minutes. The supernatant was filtered through a 0.45 \hat{ }m Corning Costar Spin-X centrifuge tube (Millipore Sigma) at 2000×g for 10 minutes. For all direct comparison experiments, plasma was first pooled and 1 mL used per EV isolation.

### Simoa Assays:

Simoa assays for CD63, CD81 and albumin were performed as previously described (Ter-Ovanesyan, Norman, et al., 2021). Due to antibody availability, CD9 ab263024 (Abcam) was used as a capture antibody instead of ab195422 (Abcam). For ApoB, mab4124 (R&D systems) was used as the capture antibody, mab41242 (R&D systems) was used as the detector antibody, and ApoB-100 BA1030 (Origene) protein was used as a standard. For SEC, onboard dilution was performed with 4×dilution for each of the assays, with an additional 4× off-board dilution for CD9 and 10× off-board dilution for ApoB. For measuring protein levels in total plasma, each protein was measured with 4× ob board dilution and three additional off-board dilutions: for CD9-40×, 80× and 160×; for CD63 and CD81 3×, 9×, 27×; for AlbuminlOOX, 3000× and 9000×; and for ApoB: 100×, 300× and 900× dilution. All samples were measured in duplicate using the HD-X analyzer (Quanterix). Tetraspanins were measured with a two-step assay, while Albumin and ApoB were measured with a three-step assay. Average Enzyme per Bead (AEB) values were calculated by the HD-X software.

### Validation of ApoB100 Simoa Assay:

Antibodies were first cross-tested using serial dilutions of protein standards. The antibody pair with the highest signal-to-background ratio was chosen. The assay was validated using dilution linearity and spike and recovery experiments. Plasma samples were diluted serially in the assay-specific buffer, a dilution factor in the middle of the linear range was chosen to be the dilution factor for the spike and recovery test. Three protein concentrations were spiked into the diluted plasma from the top calibrator used in the calibration curve. All recoveries fell in the range of 85-100% (FIG. 11). The assay validation was conducted using commercially available plasma samples

### Preparation of Columns:

Sepharose CL-2B, Sepharose CL-4B, and Sepahrose CL-6B were washed with PBS in a glass bottle. The volume of resin was washed three times with an equal volume of PBS before use. Econo-Pac Chromatography columns (Bio-Rad) were packed with resin and a frit was inserted into the column above the resin. For all columns in FIGS. 1-3 and FIGS. 8A-8E, each column was washed twice with 5 mL PBS (total of 10 mL) prior to loading of sample. For SEC columns, resin was added until the bed volume (resin without liquid) reached 10 mL. For DMC columns, Fractogel EMD-SO₃⁻ (M) (MilliporeSigma) was added as a bottom layer with 2 mL bed volume, and 10 ml of Sepharose CL-6B bed volume was added as a top layer. For TMC columns, a 2:1 by volume mixture was prepared of Fractogel EMD-SO₃⁻ (M) (MilliporeSigma) and Capto Core 700 (Cytiva) and 2 mL bed volume bottom layer was added to the column before 10 ml of Sepharose CL-6B bed volume was added as a top layer.

### Collection of Column Fractions:

Sample was loaded once PBS had finished dripping. PBS was then added in volumes equal to those being collected for one fraction of a set of fractions. And once the sample fully entered the column, 0.5 mL fractions were collected. In experiments where just the EV fractions were collected, fractions 7-10 were collected for SEC and fractions 9-12 were collected for DMC and TMC.

### Density Gradient Centrifugation:

Density gradient centrifugation (DGC) was performed as previously described. Four layers of OptiPrep (iodixanol) were prepared and stacked in a 13.2 mL polypropylene tubes (Beckman Coulter) from bottom to top: 3 mL 40%, 3 mL 20%, 3 mL 10%, 2 mL 5%. OptiPrep (MilliporeSigma) was diluted in a solution of 0.25M sucrose (MilliporeSigma) and pH 7.4 Tris-EDTA (MilliporeSigma). Sample was loaded on top of the gradient and centrifuged at 100,000 RCF for 18 hours at 4° C. After centrifugation, fractions were removed from the top 1 mL at a time.

### Negative Staining and TEM Imaging:

Carbon-coated grids (CF-400CU, Electron Microscopy Sciences) were glow discharged, and 5 μl of the sample was absorbed to the grid for 1 min. Excess sample was blotted with a Whatmann paper. The grid was then stained with 5 μl 1% Uranyl Acetate for 15 second and excess stain was blotted. Samples were imaged on a JEOL 1200EX-80 kV transmission electron microscope with an AMT 2k CCD camera.

### Mass Spectrometry:

EVs were isolated from plasma using TMC columns with a 2 mL bed volume bottom layer of 2:1 of Fractogel EMD-SO₃⁻ (M) (MilliporeSigma) to Capto Core 700 (Cytiva) and 10 mL bed volume top layer of Sepharose CL-6B (Cytiva). EVs were concentrated using Amicon Ultra-2 centrifugal 10 kD filter (MilliporeSigma). After concentration, EV protein wa precipitated by adding 9 volumes of 100% Ethanol to 1 volume of EVs, vortexing and leaving at −20C for 30 min. Sample was then centrifuged at 16,000×g for 15 minutes at 4C. Supernatant was removed and pellet was left to air dry for 10 minutes. Sample was then sent to Bruker for proteomics analysis. Sample was resuspended in 50 mM Triethylammonium bicarbonate (ThermoFisher Scientific) and digested for 2 hours at 50C using Trypsin Platinum (Promega) using 1:50 Trypsin to sample ratio by mass. After evaporating solution in a Vacufuge (Eppendorf) ar 45C, sample was resolubilized in 10 μl 0.1% Formic Acid (ThermoFisher Scientific). Next, 1.5 μl of sample was injected into C18 tips (Evosep) tips and eluted into a 25 cm length 150 μm internal diameter PepSep analytical column packed with 1.5 μm C18 beads (Dr. Maisch). Sample was eluted into a Bruker timsTOF HT. A gradient from 3% of to 28% of 0.1% Formic Acid in Acetonitrile at 63 minutes was then increased to 85% until 80 minutes. Data was analyzed using Spectronaut 17 (Biognosys) software for DIA (data independent acquisition). A false discovery rate (FDR) of 1% was used at both the peptide and protein levels.

### Example 2. Design and Preparation of Automated Device with a 24-Well Plate

The present example provides a device for running multiple SEC samples in parallel. In particular, the device is configured with a 24-well chromatography plate which can be filled with SEC resin. As a result, 24 SEC samples can be evaluated in parallel, thus increasing the throughput and reproducibility of SEC.

FIG. 14A schematically depicts a perspective view of a chromatography stand holder integrated with a liquid handling platform. FIG. 14B schematically depicts a perspective view of a chromatography plate holder including a 24-well chromatography plate. FIG. 14C schematically depicts an exploded view of a chromatography plate holder including a twenty-four well chromatography plate and a cap sized for the plate.

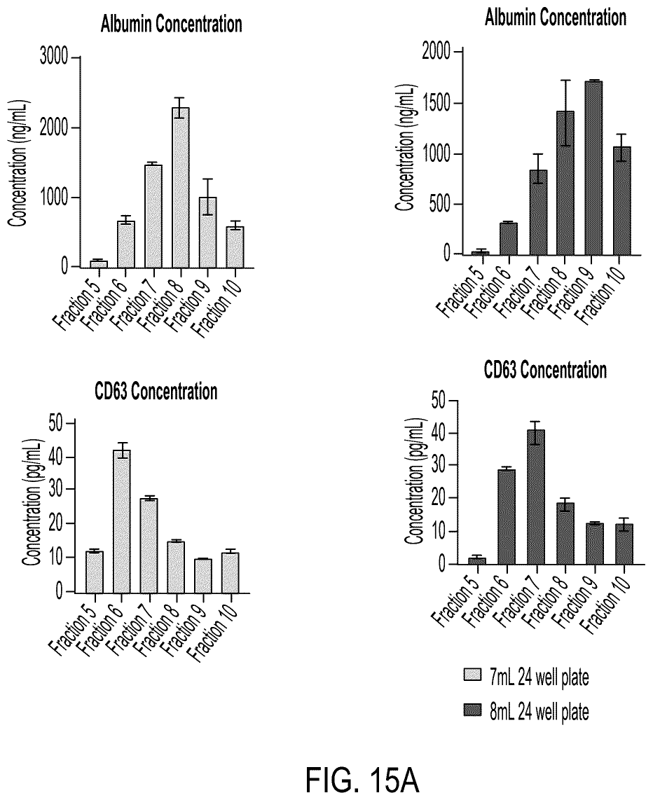

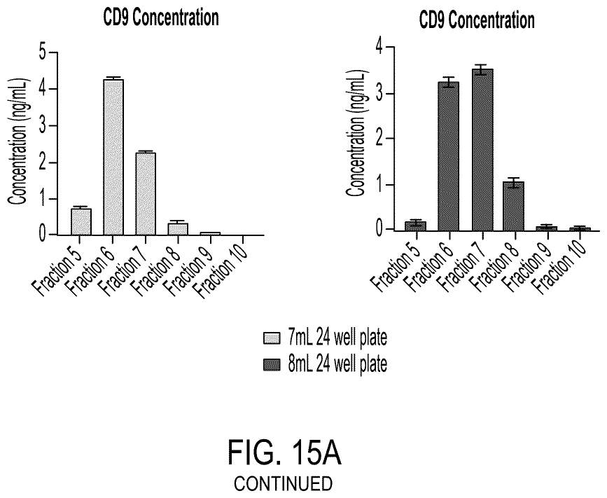

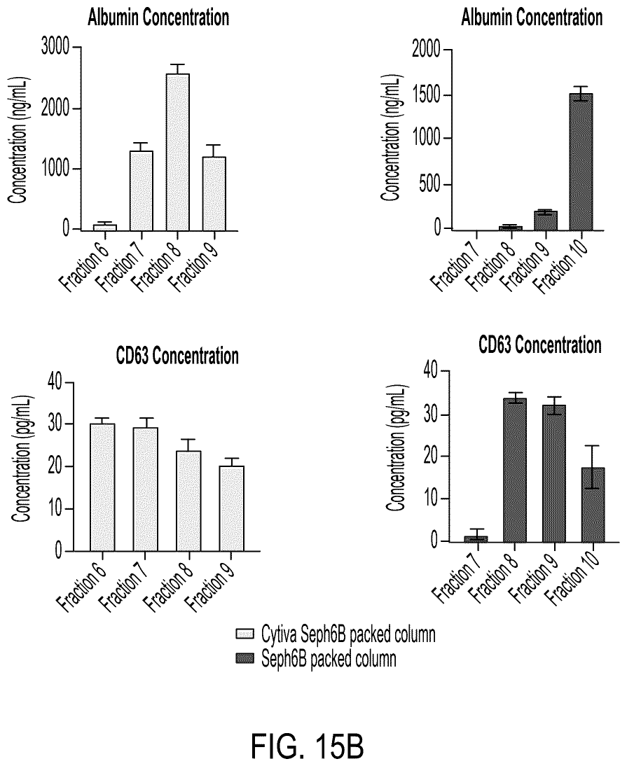

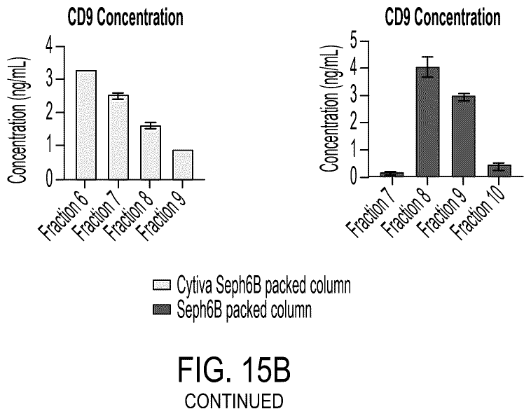

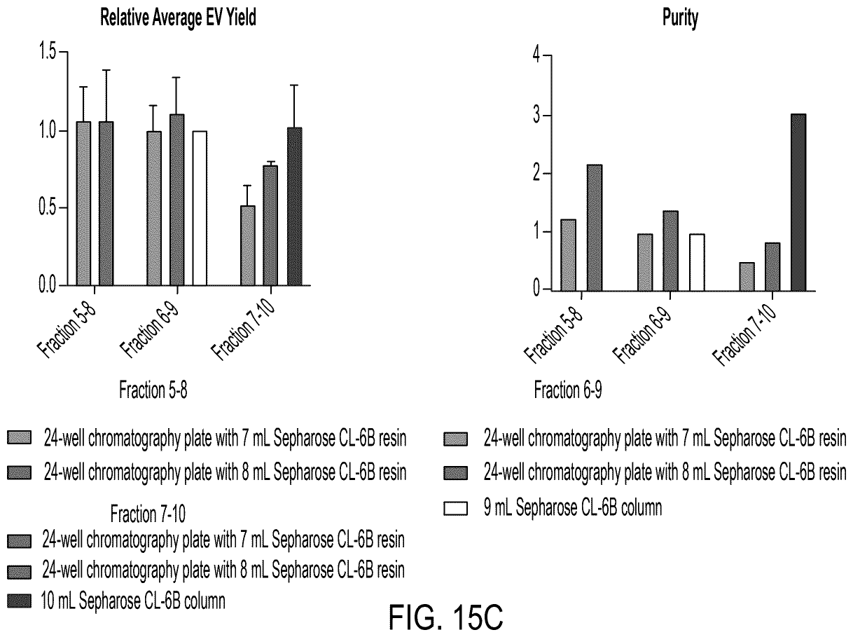

The relative EV purity and yield was compared between purification with SEC resin in 24-well plates, and SEC resin in columns. FIG. 15A depicts the levels of CD9, CD63 and albumin in fractions 5-10 for SEC in a 24-well chromatography plate with 7 or 8 mL Sepharose CL-6B resin. Simoa analysis of CD9, CD63 and albumin were performed in different fractions after loading 0.5 mL plasma into a 24-well chromatography palte with a 7 mL Sepharose CL-6B SEC resin or a 8 mL Sepharose CL-6B SEC resin. FIG. 15B depicts the levels of CD9, CD63, and albumin in fractions 5-10 for SEC with 9 mL Sepharose CL-6B resin column and 10 mL Sepharose-CL6B resin column. Simoa analysis of CD9, CD63 and albumin were performed in different fractions after loading 0.5 mL plasma into a 9 mL Sepharose CL-6B SEC resin column or a 10 mL Sepharose CL-6B SEC resin column.

The relative levels of EV yield and EV purifty was shown in FIG. 15C. Relative levels of EV yield were calculated by averaging relative levels of CD9 and CD63 for 24 well plates with 7 mL or 8 mL of Sepharose-CL6B resin to 9 mL Sepharose-CL6B column and 10 mL Sepharose-CL6B resin column. Relative EV purity was calculated by dividing EV yield by albumin levels for 24 well plates with 7 mL or 8 mL of Sepharose-CL6B resin to 9 mL Sepharose-CL6B column and 10 mL Sepharose-CL6B resin column. As demontrated in FIG. 15C, purification of EVs through the use of the device with a 24-well plate resulted in comparable yields and purity as with the Sepharose CL-6B SEC resin columns.

### References

1. Shah R, Patel T, Freedman J E. Circulating Extracellular Vesicles in Human Disease. The New England journal of medicine. 2018; 379(10):958-66.
2. Sodar B W, Kittel A, Paloczi K, Vukman K V, Osteikoetxea X, Szabo-Taylor K, et al. Low-density lipoprotein mimics blood plasma-derived exosomes and microvesicles during isolation and detection. Sci Rep. 2016; 6:24316.
3. Smolarz M, Pietrowska M, Matysiak N, Mielańczyk Ł, Widłak P. Proteome Profiling of Exosomes Purified from a Small Amount of Human Serum: The Problem of Co-Purified Serum Components. Proteomes. 2019; 7(2).
4. Johnsen K B, Gudbergsson J M, Andresen T L, Simonsen J B. What is the blood concentration of extracellular vesicles? Implications for the use of extracellular vesicles as blood-borne biomarkers of cancer. Biochim Biophys Acta Rev Cancer. 2019; 1871(1):109-16.
5. Simonsen J B. What Are We Looking At? Extracellular Vesicles, Lipoproteins, or Both? Circulation research. 2017; 121(8):920-2.
6. Hartjes T A, Mytnyk S, Jenster G W, van Steijn V, van Royen M E. Extracellular Vesicle Quantification and Characterization: Common Methods and Emerging Approaches. Bioengineering (Basel, Switzerland). 2019; 6(1).
7. Ter-Ovanesyan D, Norman M, Lazarovits R, Trieu W, Lee J H, Church G, et al. Framework for rapid comparison of extracellular vesicle isolation methods. eLife. 2021; 10.
8. Rissin D M, Kan C W, Campbell T G, Howes S C, Fournier D R, Song L, et al. Single-molecule enzyme-linked immunosorbent assay detects serum proteins at subfemtomolar concentrations. Nat Biotechnol. 2010; 28(6):595-9.
9. German J B, Smilowitz J T, Zivkovic A M. Lipoproteins: When size really matters. Curr Opin Colloid Interface Sci. 2006; 11(2-3):171-83.
10. Sniderman A D, Thanassoulis G, Glavinovic T, Navar A M, Pencina M, Catapano A, et al. Apolipoprotein B Particles and Cardiovascular Disease: A Narrative Review. JAMA Cardiol. 2019; 4(12):1287-95.
11. Monguió-Tortajada M, Gálvez-Montón C, Bayes-Genis A, Roura S, Borràs F E. Extracellular vesicle isolation methods: rising impact of size-exclusion chromatography. Cellular and Molecular Life Sciences. 2019; 76(12):2369-82.
12. Olsson U, Camejo G, Olofsson S O, Bondjers G. Molecular parameters that control the association of low density lipoprotein apo B-100 with chondroitin sulphate. Biochim Biophys Acta. 1991; 1097(1):37-44.
13. Brownlee Z, Lynn K D, Thorpe P E, Schroit A J. A novel “salting-out” procedure for the isolation of tumor-derived exosomes. J Immunol Methods. 2014; 407:120-6.
14. Van Deun J, Jo A, Li H, Lin H Y, Weissleder R, Im H, et al. Integrated Dual-Mode Chromatography to Enrich Extracellular Vesicles from Plasma. Advanced biosystems. 2020:e1900310.
15. Blom H, Åkerblom A, Kon T, Shaker S, van der Pol L, Lundgren M. Efficient chromatographic reduction of ovalbumin for egg-based influenza virus purification. Vaccine. 2014; 32(30):3721-4.
16. Norman M, Ter-Ovanesyan D, Trieu W, Lazarovits R, Kowal E J K, Lee J H, et al. L1CAM is not associated with extracellular vesicles in human cerebrospinal fluid or plasma. Nat Methods. 2021; 18(6):631-4.

### INCORPORATION BY REFERENCE

The entire disclosure of each of the patent documents, including patent application documents, scientific articles, governmental reports, websites, and other references referred to herein is incorporated by reference herein in its entirety for all purposes. In case of a conflict in terminology, the present specification controls.

The following references, to the extent that they provide exemplary procedural or other details supplementary to those set forth herein, are specifically incorporated herein by reference.

Although illustrative embodiments of the present invention have been described herein, it should be understood that the invention is not limited to those described, and that various other changes or modifications may be made by one skilled in the art without departing from the scope or spirit of the invention.

1. A device for automated chromatography of multiple samples in parallel, comprising:
a stand comprising:
a frame;
an upper portion attached to the frame, the upper portion configured to house a column comprising a resin and configured to receive a biological sample; and
a bottom portion connected to the frame, the bottom portion comprising a row of openings, each opening configured to house a tube, wherein the bottom portion may be positioned such that the row of openings is positioned to receive the biological sample from the upper portion.

2. The device of claim 1, further comprising
(a) a pump configured to deliver the biological sample; optionally, wherein the pump is a syringe pump; and/or
(b) a computer device configured to control the flow of the biological sample from the pump.

3. (canceled)

4. (canceled)

5. The device of claim 1,
(a) wherein the bottom portion is configured to house at least eight tubes;
(b) wherein the bottom portion is configured to house at least twenty-four tubes;
(c) wherein the bottom portion is configured to house at least ninety-six tubes;
(d) wherein each tube is sized to receive a sample ranging from half a milliliter to 50 milliliters;
(e) wherein the bottom portion is slideably connected to the frame; and/or
(f) wherein the biological sample is a plasma sample.

6-8. (canceled)

9. The device of claim 1, wherein the column comprises a size exclusion chromatography resin.

10. The device of claim 9,
(a) wherein the size exclusion chromatography resin is placed at a top layer of the column; and/or
(b) wherein the size exclusion chromatography resin comprises a Sepharose™ CL-6B resin.

11-15. (canceled)

16. A method of automating chromatography of multiple samples in parallel, comprising:
providing a device comprising:
a stand comprising:
a frame;
an upper portion attached to the frame, the upper portion configured to house a column comprising a resin and configured to receive a biological sample; and
a bottom portion connected to the frame, the bottom portion comprising a row of openings, each opening configured to house a tube, wherein the bottom portion may be positioned such that the row of openings is positioned to receive the biological sample from the upper portion; and
initiating a flow of the biological sample into the device, the sample comprising extracellular vesicles.

17. The method of claim 16, further comprising collecting fractions containing extracellular vesicles from the column, thereby purifying the extracellular vesicles.

18. The method of claim 16, wherein the flow is provided by a pump; optionally.
(a) wherein operation of the pump is controlled by a computer device; and/or
(b) wherein the pump is a syringe pump.

19. (canceled)

20. (canceled)

21. The method of claim 16,
(a) wherein the bottom portion is configured to house at least eight tubes;
(b) wherein the bottom portion is configured to house at least twenty-four tubes;
(c) wherein the bottom portion is configured to house at least ninety-six tubes;
(d) wherein each tube is sized to receive a sample ranging from half a milliliter to 50 milliliters;
(e) wherein the bottom portion is slideably connected to the frame; and/or
(f) wherein the biological sample is a plasma sample.

22-24. (canceled)

25. The method of claim 16, wherein the column comprises a size exclusion chromatography resin.

26. The method of claim 25,
(a) wherein the size exclusion chromatography resin is placed at a top layer of the column; and/or
(b) wherein the size exclusion chromatography resin comprises a Sepharose™ CL-6B resin.

27-31. (canceled)

32. A chromatography column for purifying extracellular vesicles from a biological sample comprising:
(a) a size exclusion chromatography resin,
(b) a cation exchange chromatography resin; and
(c) size-exclusion beads capable of capturing molecules smaller than about 700 kDa;
wherein the size exclusion chromatography resin is placed at a top layer of the column; and wherein the cation exchange chromatography resin and the size-exclusion beads are mixed and the mixture is placed at a bottom layer of the column.

33. The chromatography column of claim 32,
(a) wherein the size exclusion chromatography resin comprises a Sepharose™ CL-6B resin;
(b) wherein the cation exchange chromatography resin comprises a Fractogel® EMD-SO₃ resin; and/or
(c) wherein the size-exclusion beads comprise Capto™ Core 700 beads.

34. (canceled)

35. (canceled)

36. The chromatography column of claim 32,
(a) wherein the top layer of the column comprises about 10 mL of the size exclusion chromatography resin;
(b) wherein the bottom layer of the column comprises about 1 mL, about 2 mL, or about 4 mL of the mixture of the cation exchange chromatography resin and the size-exclusion beads; and/or
(c) wherein the cation exchange chromatography resin and the size-exclusion beads are mixed at a ratio of about 1:4, about 1:2, about 2:1, or about 4:1 by volume.

37-40. (canceled)

41. The chromatography column of claim 32, wherein the biological sample is obtained from a subject.

42. The chromatography column of claims 41, wherein the biological sample is a plasma sample or a cerebrospinal fluid (CSF) sample.

43. (canceled)

44. A method of purifying extracellular vesicles from a biological sample, the method comprising:
(a) providing a chromatography column of claim 32,
(b) introducing a biological sample comprising extracellular vesicles into the column, and
(c) collecting fractions containing extracellular vesicles from the column, thereby purifying the extracellular vesicles.

45. The device of claim 1, wherein the column comprises a size exclusion chromatography resin, and/or a cation-exchange resin.

46. The method of claim 16, wherein the column comprises a size exclusion chromatography resin, and/or a cation-exchange resin.

47. The device of claim 1, wherein the upper portion comprises the column.

48. The method of claim 16, wherein the upper portion comprises the column.

49. The device of claim 1, wherein the each opening of the bottom portion comprises the tube.

50. The method of claim 16, wherein the each opening of the bottom portion comprises the tube.

51. The device of claim 1, wherein the column comprises a cation-exchange resin and/or size exclusion beads capable of capturing molecules smaller than about 700 kDa.

52. The device of claim 52, wherein the cation exchange chromatography resin and the size-exclusion beads are mixed and the mixture is placed at a bottom layer of the column; wherein the cation exchange chromatography resin comprises a Fractogel® EMD-SO3— resin; and/or wherein the size-exclusion beads comprise Capto™ Core 700 beads.

53. The method of claim 16, wherein the column comprises a cation-exchange resin and/or size exclusion beads capable of capturing molecules smaller than about 700 kDa.

54. The method of claim 53, wherein the cation exchange chromatography resin and the size-exclusion beads are mixed and the mixture is placed at a bottom layer of the column; wherein the cation exchange chromatography resin comprises a Fractogel® EMD-SO3— resin; and/or wherein the size-exclusion beads comprise Capto™ Core 700 beads.
